(19) United States
(12) Patent Application Publication
(10) Pub. No.: US 2023/0290439 A1
(43) Pub. Date: Sep. 14, 2023

(54) A Deep Learning Framework to Identify Pathogenic Non-Coding Somatic Mutations From Personal Cancer Genomes

(71) Applicant: The Regents of the University of California, Oakland, CA (US)

(72) Inventors: Jingjing Li, San Francisco, CA (US); Cheng Wang, San Francisco, CA (US)

(21) Appl. No.: 18/016,921

(22) PCT Filed: Jul. 21, 2021
(86) PCT No.: PCT/US2021/042642

§ 371 Date: Jan. 19, 2023

Related U.S. Application Data
(60) Provisional application No. 63/055,448, filed on Jul. 23, 2020.

(51) Int. Cl. G16B 20/20; G16B 40/20; G16H 50/20

(52) U.S. Cl. CPC G16B 20/20; G16B 40/20; G16H 50/20

(57) ABSTRACT

Methods, systems, and devices, including computer programs encoded on a computer storage medium are provided for genome-wide identification of pathogenic non-coding somatic mutations associated with tumorigenesis and cancer progression. A predictive deep learning model is provided that estimates the risk of cancer progression in an individual based on detection of pathogenic non-coding somatic mutations that alter tissue-specific chromatin structure resulting in gene regulatory changes that lead to tumor formation and cancer progression.

### CROSS-REFERENCE

This application claims the benefit of U.S. Provisional Patent Application No. 63/055,448, filed Jul. 23, 2020, which application is incorporated herein by reference in its entirety.

### BACKGROUND

To date, more than 81 million simple somatic mutations (base substitutions and short indels) from cancer genomes have been catalogued by International Cancer Genome Consortium (ICGC), among which >90% fall in non-coding regions (International Cancer Genome, Hudson et al. (2010) International network of cancer genome projects. Nature, 464, 993-998). However, our current cancer genome analysis. has been largely confined to somatic mutations in coding sequences, leaving the vast majority of non-coding genome unexplored. Multiple lines of evidence have suggested a significant involvement of non-coding somatic mutations in cancer genomes: (i) it has been established that more than 90% of functional loci in complex diseases reside in the non-coding genome (Corradin, O. and Scacheri, P. C. (2014) Genome Med, 6, 85; Schaub et al. (2012) Genome Res, 22, 1748-1759), and therefore significant contribution to cancer etiologies by perturbing non-coding regulatory elements would also be expected (Khurana et al. (2016) Nat Rev Genet, 17, 93-108); (ii) specifically for cancer, individual cases studies have identified several non-coding mutations driving tumorigenesis and progression, including the well-known somatic mutations in the TERT promoter (Huang et al. (2013) Science, 339, 957-959) as well as the clustered somatic mutations affecting the cis-regulatory elements of FOXA1, which promotes prostate cancer cell proliferation (Zhou et al. (2020) Nat Commun, 11, 441); (iii) the landmark cancer epigenome study has revealed distinct chromatin architecture in tumors relative to normal tissues (Corces et al. (2018) Science, 362(6413):eaav1898), prompting further investigation on somatic mutations that alter tissue-specific chromatin structure leading to tumor formation and progression.

Despite these considerations, the recent pan-cancer genome analysis unexpectedly reported a paucity of somatic driver mutations in the non-coding genome by identifying only a handful of non-coding somatic mutations displaying significant recurrence across tumor samples (Rheinbay et al. (2020) Nature, 578, 102-111). Mutational recurrence has been used as the primary approach to infer mutational pathogenicity, which indirectly infers mutational pathogenicity based on statistical enrichment, but cannot directly assess the molecular effects of individual non-coding somatic mutations. As such, mutational recurrence analysis effectively identifies deleterious mutations forming “hotspots” in large-scale cancer genomes, but cannot capture individual pathogenic mutations in personal genomes. Given the sporadic nature of somatic mutations, it is reasonable to expect that many pathogenic mutations do not form clusters, but individually exert their effects on personal genomes. Identifying these individual mutations will not only significantly expand our view of the non-coding cancer genome, but will also foster the development of clinical tools to screen pathogenic regulatory mutations.

In addition to mutational recurrence analysis (or identifying mutation hotspots), several approaches have been proposed to individually annotate non-coding somatic mutations in cancer (as reviewed in these excellent articles (Gan, et al. (2018) Front Genet, 9, 16; Piraino et al. (2016) Ann Oncol, 27, 240-248). Some of the studies examined mutational localization in known regulatory regions, followed by motif analysis. However, many transcription factors bind to degenerate DNA sequences (Zhang et al. (2006) Nucleic Acids Res, 34, 2238-2246; Slattery et al. (2014) Trends Biochem Sci, 39, 381-399), and a single base change is more likely to be neutral than consequential. Evolutionary conservation was also integrated in the analysis; however, even if one somatic mutation affects a conserved site, the observed conservation could merely result from background selection. Some studies identified somatic mutations displaying allele-specific expression (ASE, or somatic eQTLs) in tumor samples (Cheng (2020) A catalog of cis-regulatory mutations in 12 major cancer types. bioRxiv; Zhang et al. (2018) Nat Genet, 50, 613-620; Heyn (2016) PLoS Genet, 12, e1005826). However, this association framework to identify allelic imbalance at the RNA level cannot confirm causative roles of somatic alleles. This practice also requires a large cohort of patient tumor tissues for genome and transcriptome sequencing, which is often impractical for clinical analyses. Taken together, like the mutational recurrence analysis, our current understanding of the non-coding genome in cancer has been largely derived from indirect inference from large-scale patient cohorts, which cannot directly determine the pathogenic effects of individual non-coding mutations from personal genomes. As such, in clinical practice, non-coding mutations are often excluded from clinical tumor sequencing practice and have not yet been leveraged to guide personalized screening, diagnosis and treatment.

### SUMMARY

Methods, systems, and devices, including computer programs encoded on a computer storage medium are provided for genome-wide identification of pathogenic non-coding somatic mutations associated with tumorigenesis and cancer progression. A predictive deep learning model is provided that estimates the risk of cancer progression in an individual based on detection of pathogenic non-coding somatic mutations that alter tissue-specific chromatin structure resulting in gene regulatory changes that lead to tumor formation and cancer progression.

In one aspect, a method for genome-wide identification of pathogenic non-coding somatic mutations associated with cancer is provided, the method comprising: a) providing a database comprising cancer-specific epigenomic correlation data for associations between non-coding somatic mutations and tissue-specific chromatin structural changes associated with tumorigenesis and cancer progression based on genome-wide epigenomic screening of a population of cancer patients; b) generating a deep learning model to compute the probability that a given cancer genomic sequence has an open chromatin structure; and c) using the deep learning model to identify pathogenic non-coding somatic mutations in a cancer genome, wherein a non-coding somatic mutation is considered to be pathogenic if an allelic change from its corresponding reference wild-type allele to the somatic mutation results in an alteration in predicted chromatin openness based on the deep learning model.

In certain embodiments, the deep learning model uses a convolutional neural network or a deep residual neural network.

In certain embodiments, the method further comprises calculating deep estimation from epigenome prediction (DEEP) scores (see Example 1) or DEEP+ scores (see Example 2) for each non-coding somatic mutation that is identified as pathogenic.

In certain embodiments, the cancer is prostate cancer.

In certain embodiments, the cancer is non-metastatic.

In certain embodiments, the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

In certain embodiments, the non-coding somatic mutations comprise at least one insertion, deletion, or single-nucleotide variant.

In certain embodiments, the epigenomic correlation data comprises assay for transposase-accessible chromatin sequencing (ATAC-Seq) data.

In another aspect, a method of predicting risk of tumorigenesis or cancer progression in an individual is provided, the method comprising: a) obtaining a biological sample suspected of comprising cancerous or premalignant cells from the individual; b) genotyping one or more cells in the biological sample to determine if the individual has one or more pathogenic non-coding somatic mutations; and c) calculating a DEEP score or a DEEP+ score for the pathogenic non-coding somatic mutations detected by genotyping, wherein the composite DEEP score or DEEP+ score indicates the risk of tumorigenesis or cancer progression in the individual.

In certain embodiments, the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

In certain embodiments, the non-coding somatic mutations comprise at least one insertion, deletion, or single-nucleotide variant.

In certain embodiments, the cancer is prostate cancer.

In certain embodiments, the cancer is non-metastatic.

In certain embodiments, the one or more pathogenic non-coding somatic mutations comprise one or more pathogenic non-coding somatic mutations selected from Table 1.

In certain embodiments, the one or more pathogenic non-coding somatic mutations comprise at least one pathogenic non-coding somatic mutation that alters regulation of a gene responsive to 5α-dihydrotestosterone (DHT). In some embodiments, the method further comprises predicting responsiveness of an individual with prostate cancer to treatment with an androgen receptor inhibitor based on identifying one or more pathogenic non-coding somatic mutations that alter regulation of a gene responsive to DHT.

In certain embodiments, the one or more pathogenic non-coding somatic mutations comprise at least one pathogenic non-coding somatic mutation in a gene selected from the group consisting of ING3, IPO11, LARP4, TSC22D1, MCL1, CUL4B, ZNF711, DIDO1, CDK8, HNRNPM, LHX2, NFKBIA, and MLLT3.

In certain embodiments, the method further comprises calculating a composite pLI score for the pathogenic non-coding somatic mutations detected in the individual by genotyping, wherein the composite DEEP score or DEEP+ score is used in combination with the composite pLI score to determine the risk of cancer progression in the individual.

In certain embodiments, genotyping comprises sequencing at least part of a genome of the one or more cancerous cells from the biological sample. In some embodiments, genotyping comprises sequencing the whole genome of the one or more cancerous cells from the biological sample.

In certain embodiments, the biological sample is a tumor biopsy, a tumor surgical specimen, or blood comprising circulating tumor cells.

In certain embodiments, the method further comprises performing medical imaging of a site of interest in an individual suspected of being cancerous, for example, by magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, or fluorescence imaging.

In certain embodiments, the method further comprises treating the individual for the cancer if the composite DEEP score or DEEP+ score and/or medical imaging indicates the individual is at risk of cancer progression. Exemplary anti-cancer treatments include, without limitation, surgery, radiation therapy, chemotherapy, hormonal therapy, immunotherapy, anti-angiogenic therapy, molecularly targeted or biologic therapy, and photodynamic therapy.

In another aspect, a database comprising DEEP scores or DEEP+ scores for a plurality of pathogenic non-coding somatic mutations associated with tumorigenesis or cancer progression is provided, wherein the DEEP scores or DEEP+ scores are calculated according to a method described herein. In some embodiments, the database comprises or consists of Deep scores or DEEP+ scores for pathogenic non-coding somatic mutations selected from Table 1.

In another aspect, a computer implemented method for predicting risk of prostate cancer progression in an individual is provided, the computer performing steps comprising: a) receiving prostate cancer genome sequencing data for an individual; b) identifying pathogenic non-coding somatic mutations present in the individual from the prostate cancer genome sequencing data, wherein the individual has a plurality of pathogenic non-coding somatic mutations selected from Table 1; c) calculating a DEEP score or a DEEP+ score for the pathogenic non-coding somatic mutations detected in the individual by genotyping using a database comprising DEEP scores or DEEP+ scores for a plurality of pathogenic non-coding somatic mutations associated with tumorigenesis or cancer progression, as described herein, wherein the composite DEEP score or DEEP+ score indicates the risk of prostate cancer progression in the individual; and d) displaying information regarding the risk of prostate cancer progression in the individual.

In certain embodiments, the computer implemented further comprises storing the information regarding the risk of prostate cancer progression for the individual in a database.

In another aspect, a system for predicting the risk of prostate cancer progression in an individual using a computer implemented method, described herein, is provided, the system comprising: a) a storage component for storing data, wherein the storage component has instructions for predicting the risk of prostate cancer progression in an individual based on analysis of the prostate cancer genome sequencing data stored therein; b) a computer processor for processing the prostate cancer genome sequencing data using one or more algorithms, wherein the computer processor is coupled to the storage component and configured to execute the instructions stored in the storage component in order to receive the inputted prostate cancer genome sequencing data and analyze the data according to the computer implemented method; and c) a display component for displaying the information regarding the risk of prostate cancer progression in the individual.

In another aspect, a non-transitory computer-readable medium comprising program instructions that, when executed by a processor in a computer, causes the processor to perform a computer implemented method for predicting risk of prostate cancer progression in an individual, as described herein, is provided.

In another aspect, a kit comprising the non-transitory computer-readable medium and instructions for predicting the risk of prostate cancer progression in an individual is provided.

In another aspect, a method of diagnosing an individual with prostate cancer is provided, the method comprising: a) genotyping the individual to determine if the individual has one or more pathogenic non-coding somatic mutations listed in Table 1; and b) calculating a composite DEEP score or DEEP+ score for the pathogenic non-coding somatic mutations detected in the individual by genotyping, wherein the composite DEEP score or DEEP+ score indicates whether the individual has prostate cancer. In certain embodiments, the method further comprises treating the individual for the prostate cancer if the composite DEEP score or DEEP+ score indicates the individual has prostate cancer.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. The regulatory landscape in the human prostate. (FIG. 1A). The genomic distribution of ATAC-Seq peaks in the prostate. (FIGS. 1B-1C) ATAC-Seq peaks in two genes associated with prostate cancer, NKX3-1 (FIG. 1B) and TP53 (FIG. 1C). ATAC-Seq peaks (blue) and their associated P-values (red) are shown. A strong peak is observed in the promoter of NKX3-1, and many ATAC-Seq peaks were also observed in TP53 intronic regions.

FIG. 2. The receiving operating characteristic curve from the deep-learning-based prediction. The area under the curve was estimated from a five-fold cross-validation.

FIGS. 3A-3D. Genome-wide identification of pathogenic non-coding somatic alleles in localized prostate cancer. (FIG. 3A) Distribution of the prediction scores for all the somatic alleles in this study. Three extreme outliers and their prediction scores are shown to represent significant regulatory mutations in promoter, intronic and exonic regions, respectively. Significant mutations were identified by adopting a threshold at the upper one percentile across all the mutations (the red bar). (FIGS. 3B-3D) Differential expression of MMGT1 (FIG. 3B), ARHGEF16 (FIG. 3C) and IPO11 (FIG. 3D) in TCGA prostate tumor samples of varying clinical grades relative to the normal tissues. Gene expression data were queried from UALCAN(23). No statistical significance was observed from the Gleason group 10 due to small sample size (N=10).

FIGS. 4A-4B. Characterizing the deleterious non-coding somatic mutations. (FIG. 4A) For the identified deleterious somatic variants, they displayed a significant elevation in dosage sensitivity measured by pLI scores. The variants localized in genic and intergenic regions were considered separately (P=1.98e-14 for genic variants, and P=4.77e-9 for intergenic variants, Wilcoxon rank-sum test). (FIG. 4B) For genes affected by the identified deleterious regulatory mutations, their expression in primary prostate tumors displayed marked down-regulation relative to the genome background (P values in red) and to the set of dosage-sensitive genes (P values in blue).

FIG. 5. The identified pathogenic somatic variants are convergent on androgen-receptor-mediated pathways. Genes affected by the identified deleterious somatic mutations displayed strong down-regulation and up-regulation after DHT simulation and Enzalutamide treatment, respectively.

FIGS. 6A-6B. Deleterious regulatory somatic mutations are predictive of adverse clinical outcomes. (FIG. 6A) Personal genome scan identified an extreme mutation carrier with excessive regulatory variants perturbing the 257 gene panel, corresponding to his extreme prostate cancer Gleason score (GS) of 9. (FIG. 6B) Across all the study subjects, individuals with mild tumors (GS=6) on average had one gene affected, compared with ˜2.5 affected genes for individuals with aggressive tumors (Gleason score>6, P=4.22e-4, Wilcoxon rank-sum test).

FIGS. 7A-7B. DEEP plus (DEEP+) model. (FIG. 7A) We substituted the building units in our DEEP framework with residual blocks that integrate the information adapted from both current and previous layers. The reconstituted DEEP+ framework is easier for optimization during model training and expands the accuracy upon multilayer accumulation compared to the previous version. (FIG. 7B) Our DEEP+ framework utilizes the identity shortcuts that connect the hidden upper layers. The identity functions are learned to represent the residual information from upper layers so that unparalleled scaled features extracted from the genome could be better integrated.

FIG. 8. DEEP+ model that incorporates residual deep learning framework is more robust for capturing complex genome context. We adapted data with different genome context including ATAC-seq, histone ChIP and transcription factor ChIP from different human tissues as the input for our DEEP+ training. After we trained both DEEP+ and DEEP models with the same iterations and validation approaches, we observed that DEEP+ demonstrated higher predicting performance on the same test sets at different chromatin accessibility scales. The DEEP+ model not only increases the accuracy of high confident chromatin structure prediction (e.g. ATAC-seq in prostate gland), but also expands the capacity of predicting chromatin structures of higher complexity and less organization (e.g. ChIP-seq of PR in placenta and H3K4me1 in liver.

### DETAILED DESCRIPTION OF EMBODIMENTS

Methods, systems, and devices, including computer programs encoded on a computer storage medium are provided for genome-wide identification of pathogenic non-coding somatic mutations associated with tumorigenesis and cancer progression. A predictive deep learning model is provided that estimates the risk of cancer progression in an individual based on detection of pathogenic non-coding somatic mutations that alter tissue-specific chromatin structure resulting in gene regulatory changes that lead to tumor formation and cancer progression.

Before the present methods, systems, and devices are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the nucleic acid” includes reference to one or more nucleic acids and equivalents thereof, e.g. polynucleotides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Biological sample. The term “sample” with respect to an individual encompasses blood, urine, and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

DNA samples, e.g. samples useful in genotyping, are readily obtained from any nucleated cells of an individual, e.g. hair follicles, cheek swabs, white blood cells, premalignant or cancerous cells from tissue, circulating tumor cells, etc., as known in the art.

The term “biological sample” encompasses a clinical sample. The types of “biological samples” include, but are not limited to: biological fluids, tissue samples, tissue obtained by surgical resection, tissue obtained by biopsy, cells in culture, cell supernatants, cell lysates, organs, bone marrow, blood, plasma, serum, saliva, urine, fine needle aspirate, lymph node aspirate, cystic aspirate, a paracentesis sample, a thoracentesis sample, and the like.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying or genotyping cells in the sample. Thus, the term “assaying” or “genotyping” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays or genotypes cells in the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a urine sample, a biopsy sample, a surgical specimen, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, etc.) as well as those in which prevention is desired (e.g., those with increased susceptibility to cancer, those suspected of having cancer, those with a risk of recurrence, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Substantially purified” generally refers to isolation of a substance (e.g., compound, molecule, agent) such that the substance comprises the majority percent of the sample in which it resides. Typically in a sample, a substantially purified component comprises 50%, preferably 80%-85%, more preferably 90-95% of the sample.

By “isolated” is meant an indicated cell, population of cells, or molecule is separate and discrete from a whole organism or is present in the substantial absence of other cells or biological macromolecules of the same type.

The terms “subject,” “individual” or “patient” are used interchangeably herein and refer to a vertebrate, preferably a mammal. By “vertebrate” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

As used herein, the term “probe” refers to a polynucleotide that contains a nucleic acid sequence complementary to a nucleic acid sequence present in the target nucleic acid analyte (e.g., at location of a somatic mutation). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

An “allele-specific probe” hybridizes to only one of the possible alleles of a gene (e.g., hybridizes at the location of a mutation) under suitably stringent hybridization conditions.

The term “primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a gene (e.g., hybridizes at the location of a mutation), and amplifies only one specific allele if it is present in a nucleic acid amplification reaction.

The term “common genetic variant” or “common variant” refers to a genetic variant having a minor allele frequency (MAF) of greater than 5%.

The term “rare genetic variant” or “rare variant” refers to a genetic variant having a minor allele frequency (MAF) of less than or equal to 5%.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma, and include cancers such as, but are not limited to, pancreatic cancer, lung cancer (non-small cell lung cancer, small cell lung cancer), gastric cancer, ovarian cancer, endometrial cancer, colorectal cancer, oral cancer, skin cancer, cholangiocarcinoma, head and neck cancer, breast cancer, ovarian cancer, melanoma, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models, such as xenograft models of human renal cell carcinoma. See, e.g., Pulkkanen et al., In Vivo (2000) 14:393-400 and Everitt et al., Toxicol. Lett. (1995) 82-83:621-625 for a description of animal models.

### Methods

Method are provided for genome-wide identification of pathogenic non-coding somatic mutations associated with tumorigenesis and cancer progression. A predictive deep learning model is provided for identification of pathogenic non-coding somatic mutations that alter tissue-specific chromatin structure resulting in gene regulatory changes that lead to tumor formation and cancer progression. Methods are also provided for estimating the risk of tumorigenesis and cancer progression in an individual by analyzing the contributions of pathogenic non-coding somatic mutations detected in the genome of the individual, particularly in cells or tissues suspected of being premalignant or cancerous.

The method typically involves tissue-specific genotyping of an individual to identify pathogenic non-coding somatic mutations present in the genome of cells and calculating a composite DEEP score (see Example 1) or DEEP+ score (see Example 2) for the pathogenic non-coding somatic mutations detected by genotyping, wherein the composite DEEP score or DEEP+ score indicates whether the individual is at risk of tumorigenesis and cancer progression. Cells of interest for genotyping and analysis according to the subject methods include precancerous (e.g., benign), malignant, pre-metastatic, metastatic, and non-metastatic cells.

A deep learning model is used to evaluate the effect of each somatic mutation on chromatin openness compared to a reference allele. In some embodiments, the deep learning model uses a convolutional neural network and/or a deep residual neural network. A somatic allele is considered deleterious if an allelic change from a reference allele (e.g., in the cellular genome of normal healthy tissue of the individual) to the somatic allele results in an alteration of the predicted chromatin status. For each somatic mutation, a DEEP score or DEEP+ score is used to quantify the overall allelic impact on chromatin openness. The chromatin status of a given genomic region can be predicted using the deep learning model based on the sequence of somatic alleles in the region by calculating a composite DEEP score or DEEP+ score for the somatic alleles.

Additionally, a database is provided comprising DEEP scores or DEEP+ scores for a plurality of pathogenic non-coding somatic mutations associated with tumorigenesis or cancer progression, wherein the DEEP scores or DEEP+ scores are calculated using the predictive deep learning model as described further below (e.g., see Examples). In certain embodiments, the database comprises or consists of DEEP scores or DEEP+ scores for pathogenic non-coding somatic mutations selected from Table 1.

The methods described herein are useful for identifying individuals in need of close monitoring and treatment for cancer. Individuals at high risk of tumorigenesis and cancer progression may be monitored more frequently for the development of tumors and signs of cancer progression including increases in tumor size, increases in the number of cancerous cells or tumors, cancer cell infiltration into peripheral organs, and tumor metastasis.

The methods described herein may be combined with medical imaging methods to confirm a cancer diagnosis and evaluate whether a tumor is shrinking or growing. Further, the extent of cancerous disease (how far and where the cancer has spread) can be determined to aid in determining prognosis and evaluating optimal strategies for treatment. In certain embodiments, medical imaging is performed on a site of interest in an individual, for example, by magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, or fluorescence imaging.

In addition, the methods described herein may be useful for determining that an individual should be administered an anti-cancer therapy. In certain embodiments, an anti-cancer therapy is administered to a patient if an individual is identified as being at risk of tumorigenesis and cancer progression by the methods described herein, and medical imaging indicates that cancerous cells are present. Treatment may include treating existing tumors or preventing cancer progression. An anti-cancer regimen may comprise one or more anti-cancer therapies. Examples of anti-cancer therapies include, without limitation, surgery, radiation therapy, chemotherapy, hormonal therapy, immunotherapy, anti-angiogenic therapy, molecularly targeted or biologic therapy, and photodynamic therapy.

For example, treatment may include chemotherapy with one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In another example, treatment may include targeted therapy with one or more small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

In a further example, treatment may include immunotherapy, including, but not limited to, using any of the following: a cancer vaccine (e.g., E75 HER2-derived peptide vaccine, nelipepimut-S(NeuVax), Sipuleucel-T), antibody therapy (e.g., Trastuzumab, Ado-trastuzumab emtansine, Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy.

In a further example, treatment may include radiation therapy with a radioisotope, including, but not limited to, iodine-131, strontium-89, samarium-153, and radium-223. In addition, radiation therapy may be combined with administration of a radiosensitizing drug such as, but not limited to, Cisplatin, Nimorazole, and Cetuximab.

For prostate cancer, patients with elevated prostate-specific antigen (PSA), who do not have systemic disease, may be treated with localized adjuvant therapy (e.g., radiation therapy of the prostate bed+/−pelvis lymph nodes) or a short course of anti-androgen therapy. Examples of therapeutic agents that can be used in androgen deprivation therapy include, but are not limited to, luteinizing hormone-releasing hormone agonists and antagonists such as leuprolide, goserelin, triptorelin, histrelin, buserelin, and degarelix; CYP17 inhibitors such as abiraterone, ketoconazole, orteronel, galeterone, and seviteronel; anti-androgens such as cyproterone acetate, enzalutamide, apalutamide, flutamide, bicalutamide, and nilutamide; and other androgen-suppressing agents such as estrogen and derivatives and analogues thereof. Alternatively or additionally, androgen deprivation therapy may include surgical castration (i.e., orchiectomy) to remove the testicles where androgens are produced. Patients with systemic disease after prostatectomy or radiation therapy may be further treated with chemotherapy (e.g., docetaxel, mitoxantrone and prednisone), systemic radiation therapy (e.g., samarium or strontium) and/or ADT such as anti-androgen therapy (e.g., surgical castration, finasteride, dutasteride).

### Genotyping

Individuals may be genotyped to detect pathogenic non-coding somatic mutations by any convenient method known in the art. Pathogenic non-coding somatic mutations may include common or rare genetic variants, such as mutations (e.g., nucleotide replacements, insertions, or deletions) in an intronic genomic region, a promoter, a 5′ untranslated region (5′ UTR), a 3′ untranslated region (3′ UTR), an exonic genomic region, an intergenic genomic region, or a genomic region encoding a non-coding RNA. In certain embodiments, the pathogenic non-coding somatic mutations are single nucleotide variants. In some embodiments, the non-coding somatic mutations are in dosage-sensitive genes.

For genetic testing, a biological sample containing nucleic acids is collected from an individual. The biological sample can be any sample from bodily fluids, tissue or cells that contains genomic DNA or RNA of the individual. In some embodiments, the biological sample comprises cells or tissue of interest suspected of being cancerous or premalignant such as a tumor biopsy, tumor surgical specimen, or a blood sample comprising circulating tumor cells. In certain embodiments, nucleic acids from the biological sample are isolated, purified, and/or amplified prior to analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

Detection of a mutation can be direct or indirect. For example, the mutated DNA itself can be detected directly. Alternatively, the mutation can be detected indirectly from cDNAs, amplified RNAs or DNAs, or proteins expressed by a mutated allele. Any method that detects a base change in a nucleic acid sample or an amino acid change in a protein can be used. For example, allele-specific probes that specifically hybridize to a nucleic acid containing the mutated sequence can be used to detect the mutation. A variety of nucleic acid hybridization formats are known to those skilled in the art. For example, common formats include sandwich assays and competition or displacement assays. Hybridization techniques are generally described in Hames, and Higgins “Nucleic Acid Hybridization, A Practical Approach,” IRL Press (1985); Gall and Pardue, Proc. Natl. Acad. Sci. U.S.A., 63:378-383 (1969); and John et al Nature, 223:582-587 (1969).

Sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex.

In one embodiment, the allele-specific probe is a molecular beacon. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence.

For detection, the molecular beacon is designed to only emit fluorescence when bound to a specific allele of a gene. When the molecular beacon probe encounters a target sequence with as little as one non-complementary nucleotide, the molecular beacon preferentially stay in its natural hairpin state and no fluorescence is observed because the fluorophore remains quenched. See, e.g., Nguyen et al. (2011) Chemistry 17(46):13052-13058; Sato et al. (2011) Chemistry 17(41):11650-11656; Li et al. (2011) Biosens Bioelectron. 26(5):2317-2322; Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

In another embodiment, detection of the mutated sequence is performed using allele-specific amplification. In the case of PCR, amplification primers can be designed to bind to a portion of one of the disclosed genes, and the terminal base at the 3′ end is used to discriminate between the major and minor alleles or mutant and wild-type forms of the genes. If the terminal base matches the major or minor allele, polymerase-dependent three prime extension can proceed. Amplification products can be detected with specific probes. This method for detecting point mutations or polymorphisms is described in detail by Sommer et al. in Mayo Clin. Proc. 64:1361-1372 (1989).

Tetra-primer ARMS-PCR uses two pairs of primers that can amplify two alleles of a gene in one PCR reaction. Allele-specific primers are used that hybridize at the location of the mutated sequence, but each matches perfectly to only one of the possible alleles. If a given allele is present in the PCR reaction, the primer pair specific to that allele will amplify that allele, but not the other allele of the gene. The two primer pairs for the different alleles may be designed such that their PCR products are of significantly different length, which allows them to be distinguished readily by gel electrophoresis. See, e.g., Munoz et al. (2009) J. Microbiol. Methods. 78(2):245-246 and Chiapparino et al. (2004) Genome. 47(2):414-420; herein incorporated by reference.

Mutations in a gene may also be detected by ligase chain reaction (LCR) or ligase detection reaction (LDR). The specificity of the ligation reaction is used to discriminate between the major and minor alleles of a gene. Two probes are hybridized at the site of the mutation in a nucleic acid of interest, whereby ligation can only occur if the probes are identical to the target sequence. See e.g., Psifidi et al. (2011) PLoS One 6(1):e14560; Asari et al. (2010) Mol. Cell. Probes. 24(6):381-386; Lowe et al. (2010) Anal Chem. 82(13):5810-5814; herein incorporated by reference.

As another example, an array comprising probes for detecting mutant alleles can be used. For example, SNP arrays are commercially available from Affymetrix and Illumina, which use multiple sets of short oligonucleotide probes for detecting known SNPs. The design of SNP arrays, such as manufactured by Affymetrix or Illumina, is described further in LaFamboise, “Single nucleotide polymorphism arrays: a decade of biological, computational and technological advances,” Nuc. Acids Res. 37(13):4181-4193 (2009).

Another method that can be used for detection of mutant alleles is PCR-dynamic allele specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tm) that can be readily detected.

A variety of other techniques can be used to detect mutations, including but not limited to, the Invader assay with Flap endonuclease (FEN), the Serial Invasive Signal Amplification Reaction (SISAR), the oligonucleotide ligase assay, restriction fragment length polymorphism (RFLP), single-strand conformation polymorphism, temperature gradient gel electrophoresis (TGGE), and denaturing high performance liquid chromatography (DHPLC). See, for example Molecular Analysis and Genome Discovery (R. Rapley and S. Harbron eds., Wiley 1^{st} edition, 2004); Jones et al. (2009) New Phytol. 183(4):935-966; Kwok et al. (2003) Curr. Issues Mol. Biol. 5(2):43-60; Munoz et al. (2009) J. Microbiol. Methods. 78(2):245-246; Chiapparino et al. (2004) Genome. 47(2):414-420; Olivier (2005) Mutat. Res. 573(1-2):103-110; Hsu et al. (2001) Clin. Chem. 47(8):1373-1377; Hall et al. (2000) Proc. Natl. Acad. Sci. U.S.A. 97(15):8272-8277; Li et al. (2011) J. Nanosci. Nanotechnol. 11(2):994-1003; Tang et al. (2009) Hum. Mutat. 30(10):1460-1468; Chuang et al. (2008) Anticancer Res. 28(4A):2001-2007; Chang et al. (2006) BMC Genomics 7:30; Galeano et al. (2009) BMC Genomics 10:629; Larsen et al. (2001) Pharmacogenomics 2(4):387-399; Yu et al. (2006) Curr. Protoc. Hum. Genet. Chapter 7: Unit 7.10; Lilleberg (2003) Curr. Opin. Drug Discov. Devel. 6(2):237-252; and U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for a description of such methods; herein incorporated by reference in their entireties.

In certain embodiments, a probe set is used, wherein the probe set comprises a plurality of allele-specific probes for detecting pathogenic non-coding somatic mutations in the subject's genome. The probe set may comprise one or more allele-specific polynucleotide probes. An allele-specific probe hybridizes to only one of the possible alleles of a gene under suitably stringent hybridization conditions. Individual polynucleotide probes comprise a nucleotide sequence derived from the nucleotide sequence of the target mutated allele sequences or complementary sequences thereof. The nucleotide sequence of the polynucleotide probe is designed such that it corresponds to, or is complementary to the target mutated allele sequences. The allele-specific polynucleotide probe can specifically hybridize under either stringent or lowered stringency hybridization conditions to a region of the target mutated allele sequences, to the complement thereof, or to a nucleic acid sequence (such as a cDNA) derived therefrom.

The selection of the allele-specific polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the allele-specific polynucleotide probe is complementary to the region of a single mutated allele target DNA or mRNA sequence. Computer programs can also be employed to select allele-specific probe sequences that may not cross hybridize or may not hybridize non-specifically.

The allele-specific polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The allele-specific polynucleotide probes of a probe set can comprise RNA, DNA, RNA or DNA mimetics, or combinations thereof, and can be single-stranded or double-stranded. Thus, the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In another embodiment, a set of allele-specific primers is used, wherein the set of allele-specific primers comprises a plurality of allele-specific primers for detecting pathogenic non-coding somatic mutations in the subjects genome. An allele-specific primer matches the sequence exactly of only one of the possible somatic alleles, hybridizes at the location of the pathogenic non-coding somatic mutation, and amplifies only one specific mutated allele if it is present in a nucleic acid amplification reaction. For use in amplification reactions such as PCR, a pair of primers can be used for detection of a mutated allele sequence. Each primer is designed to hybridize selectively to a single allele at the site of the mutation in the gene under stringent conditions, particularly under conditions of high stringency, as known in the art. The pairs of allele-specific primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative FOR-based assays for SNP genotyping of subjects. Alternatively, these primers may be used in combination with probes, such as molecular beacons in amplifications using real-time PCR.

A label can optionally be attached to or incorporated into an allele-specific probe or primer polynucleotide to allow detection and/or quantitation of a target mutated allele sequence. The target mutated polynucleotide may be from genomic DNA, expressed RNA, a cDNA copy thereof, or an amplification product derived therefrom, and may be the positive or negative strand, so long as it can be specifically detected in the assay being used. Similarly, an antibody may be labeled that detects a polypeptide expression product of the mutated allele.

In certain multiplex formats, labels used for detecting different mutant alleles may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Detectable labels useful in the practice of the invention may include any molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neon, G418^{r}) dihydrofolate reductase (DHFR), hygromycin-B-phosphotransferase (HPH), thymidine kinase (TK), 3-galactosidase (lacZ), and xanthine guanine phosphoribosyltransferase (XGPRT), beta-glucuronidase (gus), placental alkaline phosphatase (FLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include isotopic labels, including radioactive and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹¹³P, ⁵²Fe, ⁶²Cu, ⁶⁴Cu, ⁶⁷Cu, ⁶⁷Ga, ⁶⁸Ga, ⁸⁶Y, ⁹⁹Y, ⁸⁹Zr, ⁹⁴mTC, ⁹⁴TC, ⁹⁹mTC, ¹⁶⁴Gd, ¹⁶⁶Gb, ¹⁶⁶Gd, ¹⁶⁷Gd, ¹⁶⁸Gd, ¹⁵O, ¹⁸⁶Re, ¹⁸⁸Re, ⁵¹M, ⁵²Mn, ⁵⁵Co, ⁷²As, ⁷⁶Br, ⁷⁶Br, ^{82m}Rb, and ⁸³Sr. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The terms also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate). As with many of the standard procedures associated with the practice of the invention, skilled artisans will be aware of additional labels that can be used.

Genotyping may also comprise sequencing nucleic acids from a sample collected from an individual using any convenient sequencing protocol. Sequencing platforms that can be used include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Genetic testing services exist, which provide full genome sequencing using massively parallel sequencing. Massively parallel sequencing is described e.g. in U.S. Pat. No. 5,695,934, entitled “Massively parallel sequencing of sorted polynucleotides,” and US 2010/0113283 A1, entitled “Massively multiplexed sequencing.” Massively parallel sequencing typically involves obtaining DNA representing an entire genome, fragmenting it, and obtaining millions of random short sequences, which are assembled by mapping them to a reference genome sequence. Commercial services are available that are capable of genotyping approximately 1 million sequences for a fixed fee.

Genetic analysis can be carried out with a variety of methods that do not involve massively parallel random sequencing. For example, a commercially available MassARRAY system can be used. This system uses matrix-assisted laser desorption ionization time-of-flight mass spectrometry (MALDI-TOF MS) coupled with single-base extension PCR for high-throughput multiplex detection of mutations. Another commercial system, the Illumina Golden Gate assay, generates mutation-specific PCR products that are subsequently hybridized to beads either on a solid matrix or in solution. Three oligonucleotides are synthesized for each mutant: two allele specific oligonucleotides (ASOs) that distinguish the mutated sequence, and a locus specific sequence (LSO) just downstream of the mutation site. The ASO and LSO sequences also contain target sequences for a set of universal primers, while each LSO also contains a particular address sequences (the “illumicode”) complementary to sequences attached to beads.

### Data Analysis

In some embodiments, one or more pattern recognition methods can be used in automating analysis of genetic data and generating a predictive model. The predictive models and/or algorithms can be provided in a machine-readable format and may be used to correlate pathogenic non-coding somatic mutations identified in a patient by genotyping with the risk of tumorigenesis and cancer progression. Generating the predictive model may comprise, for example, the use of an algorithm or classifier. In some embodiments, a deep learning model based on a deep convolution neural network (CNN) and/or a deep residual neural network is used to predict tissue-specific chromatin structure for a given genomic sequence and to identify somatic mutations that alter the chromatin structure in a deleterious manner that promotes tumorigenesis and cancer progression. The deep learning model can be used for genome-wide computation of DEEP scores or DEEP+ scores for somatic mutations (see Examples).

### System and Computer Implemented Methods for Predicting the Risk of Cancer Progression

In a further aspect, the invention includes a computer implemented method for predicting the risk of tumorigenesis or cancer progression in an individual. The computer performs steps comprising a) receiving genome sequencing data for an individual; b) identifying pathogenic non-coding somatic mutations present in the genome of the individual from the genome sequencing data; c) calculating a composite deep estimation from epigenome prediction (DEEP) score or DEEP+ score for the pathogenic non-coding somatic mutations detected in the individual by genotyping using a database comprising DEEP scores or DEEP+ scores for the pathogenic non-coding somatic mutations; and d) displaying information regarding the risk of cancer progression in the individual. In certain embodiments, the database comprises DEEP scores or DEEP+ scores for a plurality of pathogenic non-coding somatic mutations selected from Table 1. In certain embodiments, the computer implemented method further comprises storing the information regarding the risk of cancer progression in the individual in a database.

The method can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, a system for performing the computer implemented method, as described, is provided. Such a system includes a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component includes instructions for predicting the risk of cancer progression in the individual based on analysis of genomic sequencing data stored therein. The computer processor is coupled to the storage component and configured to execute the instructions stored in the storage component in order to receive genome sequencing data and analyze the data according to one or more algorithms (e.g., deep convolutional neural network or deep residual neural network), as described herein. The display component displays information regarding the risk of cancer progression in the individual.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

### Kits

Kits are also provided for carrying out the methods described herein. In some embodiments, the kit comprises software for carrying out the computer implemented methods for predicting the risk of cancer progression in an individual based on detection of pathogenic non-coding somatic mutations, as described herein. In some embodiments, the kit further comprises a container for collecting a DNA sample from an individual. The kit may also include reagents for purifying and/or sequencing a DNA sample.

In addition, the kits may further include (in certain embodiments) instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The methods described herein are useful for predicting risk of tumorigenesis and cancer progression in an individual based on personalized tissue-specific genotyping to detect pathogenic non-coding somatic mutations and may also aid in selection of an appropriate treatment regimen. The methods are applicable to patients having cancer, including, but not limited to, prostate cancer, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method for genome-wide identification of pathogenic non-coding somatic mutations associated with cancer, the method comprising:
   a) providing a database comprising cancer-specific epigenomic correlation data for associations between non-coding somatic mutations and tissue-specific chromatin structural changes associated with tumorigenesis and cancer progression based on genome-wide epigenomic screening of a population of cancer patients;
   b) generating a deep learning model to compute the probability that a given cancer genomic sequence has an open chromatin structure; and
   c) using the deep learning model to identify pathogenic non-coding somatic mutations in a cancer genome, wherein a non-coding somatic mutation is considered to be pathogenic if an allelic change from its corresponding reference wild-type allele to the somatic mutation results in an alteration in predicted chromatin openness based on the deep learning model.
2. The method of aspect 1, wherein the deep learning model uses a convolutional neural network or a deep residual neural network.
3. The method of aspect 2, further comprising calculating a deep estimation from epigenome prediction (DEEP) score or a DEEP+ score for each non-coding somatic mutation that is identified as pathogenic.
4. The method of any one of aspects 1 to 3, wherein the cancer is prostate cancer.
5. The method of any one of aspects 1 to 4, wherein the cancer is non-metastatic.
6. The method of any one of aspects 1 to 5, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.
7. The method of any one of aspects 1 to 6, wherein the non-coding somatic mutations comprise at least one insertion, deletion, or single-nucleotide variant.
8. The method of any one of aspects 1 to 7, wherein the epigenomic correlation data comprises assay for transposase-accessible chromatin sequencing (ATAC-Seq) data.
9. A method of predicting risk of tumorigenesis or cancer progression in an individual, the method comprising:
   a) obtaining a biological sample suspected of comprising cancerous or premalignant cells from the individual;
   b) genotyping one or more cells in the biological sample to determine if the individual has one or more pathogenic non-coding somatic mutations; and
   c) calculating a composite deep estimation from epigenome prediction (DEEP) score or a DEEP+ score for the pathogenic non-coding somatic mutations detected by genotyping, wherein the composite DEEP score or DEEP+ score indicates the risk of tumorigenesis or cancer progression in the individual.
10. The method of aspect 9, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.
11. The method of aspect 9 or 10, wherein the non-coding somatic mutations comprise at least one insertion, deletion, or single-nucleotide variant.
12. The method of any one of aspects 9 to 11, wherein the cancer is non-metastatic.
13. The method of any one of aspects 9 to 12, wherein the cancer is prostate cancer.
14. The method of aspect 13, wherein the one or more pathogenic non-coding somatic mutations comprise one or more pathogenic non-coding somatic mutations selected from Table 1.
15. The method of any one of aspects 9 to 14, further comprising predicting responsiveness of the individual to treatment with an androgen receptor inhibitor based on identifying one or more pathogenic non-coding somatic mutations that alter regulation of a gene responsive to 5α-dihydrotestosterone (DHT).
16. The method of any one of aspects 9 to 15, wherein the one or more pathogenic non-coding somatic mutations comprise at least one pathogenic non-coding somatic mutation in a gene selected from the group consisting of ING3, IPO11, LARP4, TSC22D1, MCL1, CUL4B, ZNF711, DIDO1, CDK8, HNRNPM, LHX2, NFKBIA, and MLLT3.
17. The method of any one of aspects 9 to 16, further comprising calculating a composite pLI score for the pathogenic non-coding somatic mutations detected in the individual by genotyping, wherein the composite DEEP score or DEEP+ score is used in combination with the composite pLI score to determine the risk of prostate cancer progression in the individual.
18. The method of any one of aspects 9 to 17, wherein said genotyping comprises sequencing at least part of a genome of the one or more cancerous cells from the biological sample.
19. The method of aspect 18, wherein said genotyping comprises sequencing the whole genome of the one or more cells from the biological sample.
20. The method of any one of aspects 9 to 19, wherein the biological sample is a tumor biopsy, a tumor surgical specimen, or blood comprising circulating tumor cells.
21. The method of any one of aspects 9 to 20, further comprising performing medical imaging of a site of interest in the individual that is suspected of being cancerous, for example, by magnetic resonance imaging (MRI), positron emission tomography (PET), single photon emission computed tomography (SPECT), computed tomography (CT), ultrasound imaging (UI), optical imaging (01), photoacoustic imaging (PI), fluoroscopy, or fluorescence imaging.
22. The method of any one of aspects 9 to 21, further comprising treating the individual for the cancer if the composite DEEP score or DEEP+ score indicates the individual is at risk of cancer progression.
23. The method of aspect 22, wherein said treating comprises surgery, radiation therapy, chemotherapy, hormonal therapy, immunotherapy, anti-angiogenic therapy, molecularly targeted or biologic therapy, or photodynamic therapy, or a combination thereof.
24. A database comprising DEEP scores or DEEP+ scores for a plurality of pathogenic non-coding somatic mutations associated with tumorigenesis or cancer progression, wherein the DEEP scores or DEEP+ scores are calculated according to the method of any one of aspects 1 to 8.
25. The database of aspect 24, wherein the database comprises or consists of Deep scores or DEEP+ scores for pathogenic non-coding somatic mutations selected from Table 1.
26. A computer implemented method for predicting risk of prostate cancer progression in an individual, the computer performing steps comprising:
   a) receiving prostate cancer genome sequencing data for an individual;
   b) identifying pathogenic non-coding somatic mutations present in the individual from the prostate cancer genome sequencing data, wherein the individual has a plurality of pathogenic non-coding somatic mutations selected from Table 1;
   c) calculating a composite deep estimation from epigenome prediction (DEEP) score or a DEEP+ score for the pathogenic non-coding somatic mutations detected in the individual by genotyping using the database of aspect 25, wherein the composite DEEP score or DEEP+ score indicates the risk of prostate cancer progression in the individual; and
   d) displaying information regarding the risk of prostate cancer progression in the individual.
27. The computer implemented method of aspect 26, further comprising storing the information regarding the risk of prostate cancer progression in the individual in a database.
28. A system for predicting the risk of prostate cancer progression in an individual using the computer implemented method of aspect 26 or 27, the system comprising:
   a) a storage component for storing data, wherein the storage component has instructions for predicting the risk of prostate cancer progression in an individual based on analysis of the prostate cancer genome sequencing data stored therein;
   b) a computer processor for processing the prostate cancer genome sequencing data using one or more algorithms, wherein the computer processor is coupled to the storage component and configured to execute the instructions stored in the storage component in order to receive the inputted prostate cancer genome sequencing data and analyze the data according to the computer implemented method of aspect 26 or 27; and
   c) a display component for displaying the information regarding the risk of prostate cancer progression in the individual.
29. A non-transitory computer-readable medium comprising program instructions that, when executed by a processor in a computer, causes the processor to perform the computer implemented method of aspect 26 or 27.
30. A kit comprising the non-transitory computer-readable medium of aspect 29 and instructions for predicting the risk of prostate cancer progression in an individual.
31. A method of diagnosing an individual with prostate cancer, the method comprising:
   a) genotyping the individual to determine if the individual has one or more pathogenic non-coding somatic mutations listed in Table 1; and
   b) calculating a composite deep estimation from epigenome prediction (DEEP) score or a DEEP+ score for the pathogenic non-coding somatic mutations detected in the individual by genotyping, wherein the composite DEEP score or DEEP+ score indicates whether the individual has prostate cancer.
32. The method of aspect 31, further comprising treating the individual for the prostate cancer if the composite DEEP score or DEEP+ score indicates the individual has prostate cancer.
33. The method of aspect 32, wherein said treating comprises surgery, radiation therapy, chemotherapy, hormonal therapy, immunotherapy, anti-angiogenic therapy, molecularly targeted or biologic therapy, or photodynamic therapy, or a combination thereof.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

A Deep Learning Framework to Identify Pathogenic Non-Coding Somatic Mutations from Personal Prostate Cancer Genomes

### Introduction

We herein adopted a deep-learning based framework to mechanistically quantify the impact of every somatic allele on perturbing tissue-specific epigenomes, which has now enabled us to directly identify pathogenic non-coding somatic alleles in each personal cancer genome. We leveraged this new model to analyze somatic mutations in localized prostate cancer genomes, which serve as an excellent model based on the following considerations: (1) compared with metastatic cancers, localized prostate cancers are more likely to be affected by simple somatic mutations (base substitutions and short insertion and deletions, indels) than somatic copy number aberrations(16). However, many clinical cases cannot be explained by coding sequence mutations, and thus pathogenic non-coding somatic mutations are yet to be identified; (2) localized prostate cancers are slow-growing tumors, which allows us to use reference tissue-specific epigenomes from health individuals to study how somatic alleles could alter the reference epigenome to promote tumor formation and progression. However, metastatic prostate cancers tend to have differential epigenetics(16,17), which would complicate the analysis; (3) compared with many other cancer types, prostate cancer have a more established molecular etiology centralized on the androgen receptor (AR)-mediated signaling pathways(18,19). This knowledge will help validate the identified pathogenic non-coding variants for their roles in perturbing AR pathways; (4) because prostate cancer is the second most prevalent cancer in males, which could be cured at the localized stage and becomes lethal at the metastatic stage, expanding our analysis to the vast non-coding genome will help improve diagnostic yield in our clinical sequencing practice, fostering the development of personalized therapeutic strategies. With the new deep learning framework, we identified numerous novel pathogenic non-coding somatic mutations from personal genomes, and significantly expanded our knowledge of genes in prostate cancer. Importantly, our functional genomic analysis demonstrated that these newly identified novel somatic mutations preferentially affect genes responsive to 5α-dihydrotestosterone (DHT, the physiological androgen) stimulation and enzalutamide treatment (an androgen receptor inhibitor), not only confirming their implication in prostate cancer but also highlighting the therapeutic value of our approach. For individual patients, our analysis further demonstrated that the identified pathogenic non-coding somatic alleles in personal genomes are significant indicators of clinical outcomes of localized prostate cancer. Overall, compared with previous cohort-based analyses, our work is able to identify pathogenic non-coding somatic alleles from personal genomes, which can be deployed as a clinical tool for personalized screening and for developing personalized therapeutic strategies.

### Methods and Materials

### The Genomic Resources

We downloaded genome data and somatic mutations from ICGC (International Cancer Genome Consortium) data portal. We examined 707,610 simple somatic mutations in the localized prostate cancer (the PRAD-CA) cohort including 306 donors. Gleason scores were provided for a subset of the patients. This cohort has the largest sample sizes and uniformly benchmarked clinical information. We repurposed the HOMER software(20) to annotate the localization of each genomic mutation relative to protein-coding genes, enabling us to identify 422,314 somatic mutations that could be associated with protein-coding genes. We then annotated each somatic mutation for their localization in intronic, promoter, 5′UTR, 3′UTR, exonic and intergenic regions (close to protein-coding sequences). We retrieved the prostate ATAC-Seq data from ENCODE (21) data portal (ENCFF670GFY). Peak call was performed in the aligned BAM file using HMMRATAC(22). Peak annotation was performed by implementing HOMER(20). TCGA gene expression data (the primary prostate tumors) were queried from UALCAN(23); we also downloaded the TCGA transcriptome data for the primary prostate tumors (TCGA-PRAD) and normal prostate tissues. We implemented DESeq2(24) to compute the expression fold changes and differential expression. pLI scores were retrieved from an earlier paper(25). We retrieved RNA-Seq data in the LNCaP prostate cancer cell line after DHT stimulation and enzalutamide treatment(26)(GEO accession: GSE110903). For gene symbols mapped onto multiple Ensembl identifiers, we averaged their expression data. We also average gene expression across multiple replicates, and only considered genes with moderate or significant abundance in our comparison (FPKM>1). The human proteome map data were retrieved from the original publication(27). We averaged protein abundance for gene symbols mapped onto different protein identifiers. CADD predictions(28) and GERP++ scores(29) were retrieved from original publications. The reference genome in this study was based on GRCh37 (hg19).

### The Deep Learning Model

We developed a deep convolutional neural network with multilayers, and the network consists of alternating convolution and maxpooling layers, taking input of every 2-kb sequence to predict the associated chromatin openness in the prostate. The model was described in a previous paper (Zhou et al. (2015) Nat Methods, 12, 931-934 and Zhou et al. (2018) Nat Genet, 50, 1171-1179; herein incorporated by reference in their entireties), and we further adapted the model for our prostate cancer genome analysis. Each of our convolutional layers contains 320, 480, and 640 hidden neurons, and the output of each convolutional layer is activated by the ReLU function before propagating to the next maxpooling or convolutional layers. We implemented a fully connected layer with ReLU activation on top of the three convolutional layers, which is further propagated to the output sigmoid layer to compute the probability of a given input sequence having an open chromatin. Following the protocol in previous papers(30,31), we trained the deep learning model using the prostate ATAC-Seq peaks. Specifically, we first split the entire genome into every 200-bp bins, and we then considered a subset of the 200-bp sequences as positive samples in our model training if 50% of the sequences were overlapped with the prostate ATAC-Seq peaks. All the 200-bp sequences were padded with 900-bp sequences at both upstream and downstream regions as the context sequences, which were subsequently fed into the deep neural network for model training or testing. Similar with previous work(30,31), we adopted a holdout strategy to verify the model performance, where we randomly held out a chromosome, trained the deep learning model using all other chromosomes, and then independently tested on all the sequences from this hold-out chromosome. In our study, Chromosome 5 was randomly selected as a test set.

### In Silico Mutagenesis and the Prediction of Deleterious Mutations

For each mutation, we evaluated the altered chromatin openness from one reference allele to the somatic allele in our prostate-specific deep learning model. We used each 2 kb sequence (200-bp core sequence, plus 900 bp flanking upstream and downstream sequences) to scan the 1 kb flanking genomic regions of the mutation loci at a step size of 20 bp, obtaining 101 such sequence windows. We then derived an integrated score for each somatic mutation by weighting the distance of each mutation site to each of the 101 sliding windows. The procedure was detailed in previous publications(30,31). We used kernel density estimation to approximate the underlying score distribution of all the simple somatic alleles associated with protein coding sequences in this study, and only considered those above the genome-wide threshold (score≥5.4321, the upper one percentile across the genome) as high-confidence deleterious mutations. In our analysis of clinical outcomes, as a control set, we also designated the lowest one percentile of all the genomic mutations as benign variants.

Software Used in this Study

The model DEEP was developed by PyTorch version 3.7, and the AUROC curve was generated with scikit-learn. Statistical analysis and histograms were implemented by ggplot2 using R version 3.6.3 and MATLAB R2020a.

### Results

### Overview of Prostate Cancer Genomes and the Prostate Epigenome

We retrieved localized prostate cancer genomes from the ICGC PRAD (Prostate Adenocarcinoma)-CA cohort, totaling 707,610 simple somatic mutations (indels and single-nucleotide variants, identified from 306 patient tumor samples(1,16,17). The somatic mutations were identified by comparing the index lesion and paired blood samples from patients receiving naïve treatment at the time of sampling. Under the National Compressive Cancer Network (NCCN) guideline, the tumors were of intermediate risk (T1a, T1b, T1c, T2a, T2b and T2c). Characterization of sample information, genome sequencing platforms and technical details for variant call have been detailed in a previous publication(16,17). Among 707,610 somatic mutations (0.33% were short indels and 99.67% were single-nucleotide variants), we analyzed 422,314 that could be associated with protein-coding genes, including 224503, 178130, 5165, 397, 3592, and 5703 somatic mutations in intergenic, intronic, promoter, 5′UTR, 3′UTR and exonic regions, respectively. The remaining were either mapped onto ncRNAs or had no protein-coding genes in their proximity.

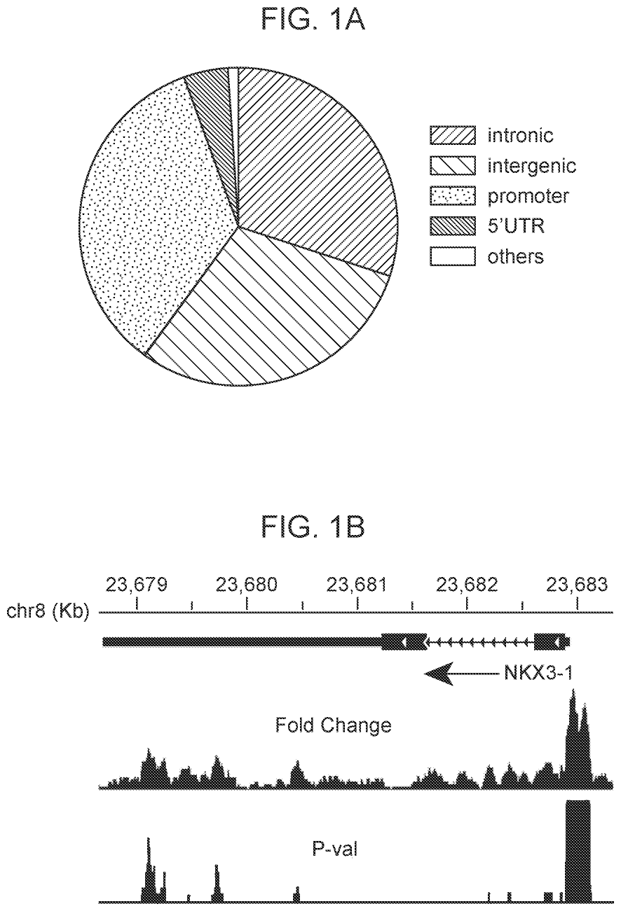

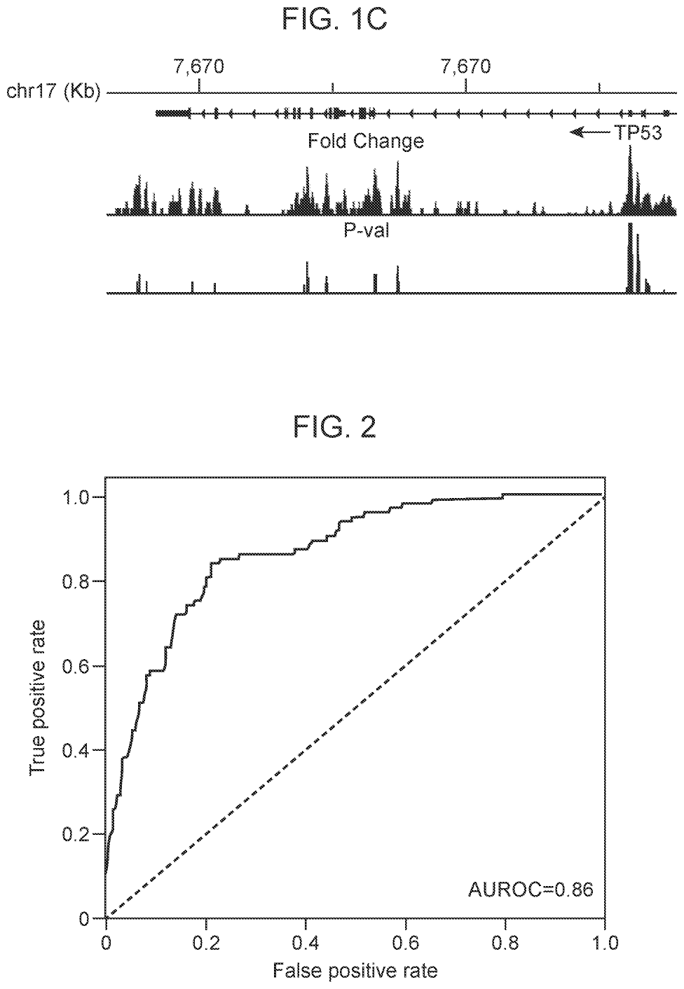

We identified deleterious non-coding mutations by evaluating their allelic effects on altering the reference epigenome in the prostate. The reference prostate epigenome was obtained from ENCODE(21), where ATAC-Seq(32) was performed on the prostate gland from a 54-year-old healthy male, revealing the regulatory landscape in the prostate. We performed peak call on this ATAC-Seq data, and identified 24,108 high-confidence elements representing a comprehensive collection of regulatory elements across the prostate genome. These elements had an average size of 411 base pairs, demonstrating a high resolution for fine-mapping non-coding variants. These elements were predominantly localized in intergenic, intronic and promoter regions (FIG. 1A). While the localization in promoter regions is expected, the prostate genome accessibility in intergenic and intronic regions suggests a widespread distribution of regulatory elements (e.g. enhancers) in these non-coding regions. We examined several known genes in prostate cancer, and ATAC-Seq clearly revealed their active regulatory elements in the prostate, including NKX3-1(33,34) and TP53(35) (FIGS. 1B-IC). Given the vast majority of somatic mutations falling in intronic and intergenic regions, it is important to determine whether their somatic alleles could perturb the prostate epigenome contributing to tumorigenesis.

### A Deep Learning Model to Predict the Prostate Epigenome

It has been established that the chromatin status of a given genomic region could be predicted, and therefore a somatic allele is considered deleterious if the allelic change from the reference allele to the somatic allele will result in an alteration of the predicted chromatin status(36,37). Machine learning approaches have been proposed to capture the allelic changes that alter the predicted chromatin structure from one allele to the other (in silico mutagenesis)(30,36,38), but have not yet been used to analyze cancer genomes, and these models were trained using heterogeneous tissue and cell types by aggregating all the ENCODE and Epigenome Roadmap resources(21,39). We herein deploy this strategy to analyzing prostate cancer genomes by training a machine learning model specific to the prostate epigenome. We trained a deep convolution neural network (CNN) to (1) predict prostate-specific chromatin structure for any given genomic sequences, and (2) to identify impactful mutations whose somatic alleles alter the predicted prostate-specific chromatin structure from the reference alleles. We developed the CNN model(6,30), and further configured the CNN model to specifically predict chromatin openness only in the prostate gland for any given 200-bp sequences. For model verification, we adopted a holdout strategy to verify the model performance, where we randomly held out a chromosome, trained the deep learning model using all other chromosomes, and then independently tested on all the sequences from this hold-out chromosome. In our study, Chromosome 5 was randomly selected as a test set, establishing the predictability of the prostate epigenome at an AUROC (area under the receiving operator characteristics) 0.86 (FIG. 2). The model was also validated using a set of randomly sampled 200-bp sequences across the genome.

With this established precision, we next mapped all the somatic mutations onto the reference genome, and asked how the somatic alleles could alter the predicted chromatin status from their respective reference alleles, and the identification of these epigenomically impactful alleles is specific to the prostate. We adopted a previously developed protocol(30) and further developed the method specific to prostate cancer genome analysis: we used sliding windows to scan through a given somatic mutation, and computed the alterations of the predicted prostate chromatin openness resulting from the allelic change for each sliding window. We averaged the predicted alterations over all the sliding windows as a composite deleteriousness score specific to the prostate gland. This score quantifies the overall allelic impact on altering chromatin architecture in the prostate. Notably, this framework does not require that a mutation should be localized in a regulatory element, and has the power to study mutations at the upstream or downstream of a given regulatory element, enabling us to model positional effects of somatic alleles. Moreover, compared with previous mutational recurrence analysis in large-scale patient cohorts, this new framework can be directly deployed to scan personal genomes. For convenience, we termed this framework DEEP (deep estimation from epigenome prediction) in our prostate cancer study.

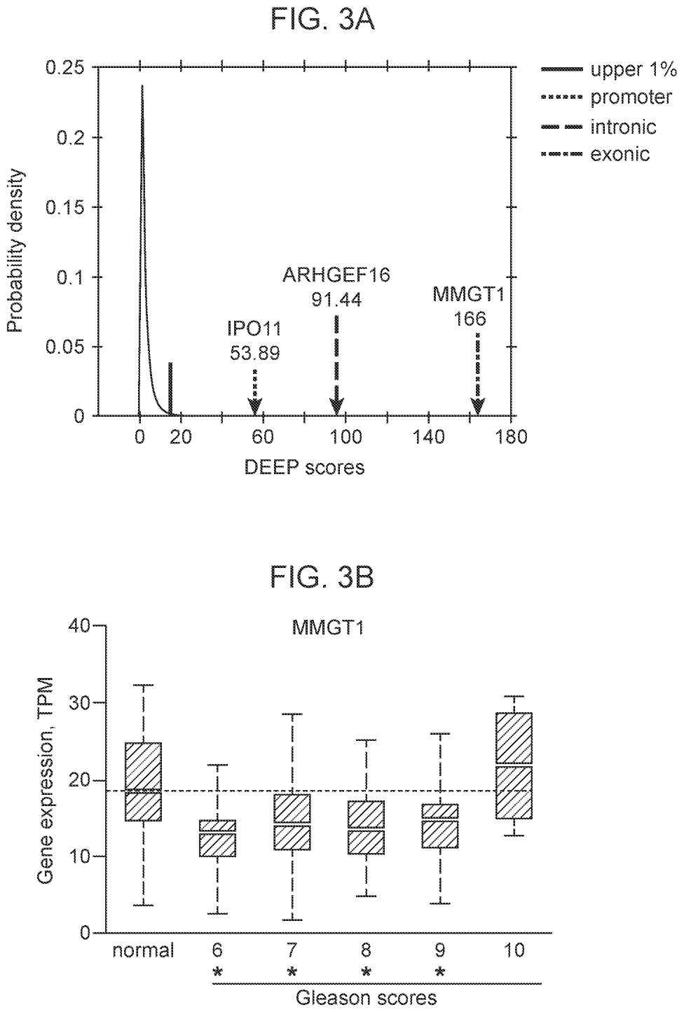

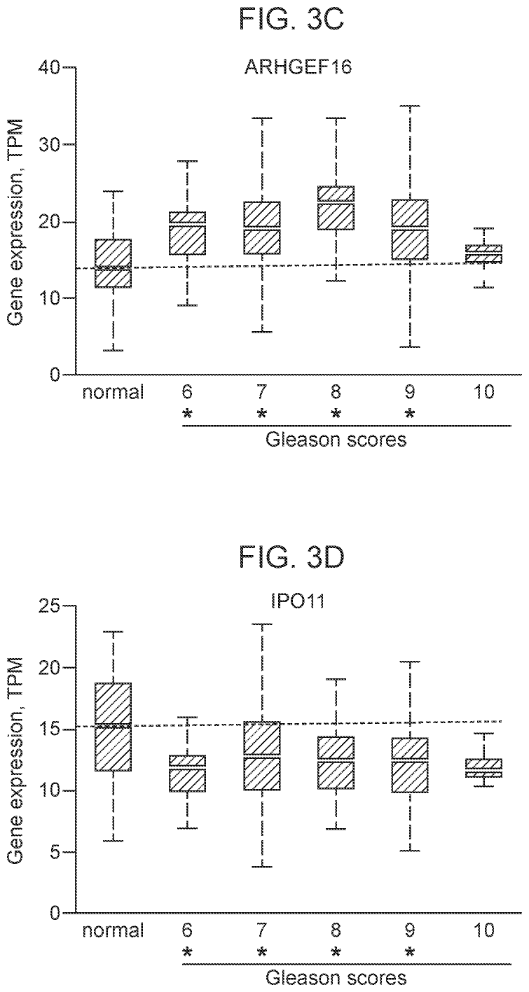

We computed DEEP scores for the entire collection of somatic mutations in this study (including those in coding sequences, and the score distribution is shown in FIG. 3A. The score distribution was peaked around 0, suggesting that the vast majority of the somatic alleles were effectively neutral in localized prostate cancer, having little effect on altering the prostate chromatin architecture. However, numerous outliers were also detected hallmarking extreme effects on altering chromatin structure by the somatic alleles. Across all the 422,314 somatic mutations analyzed, the one receiving the highest score (DEEP score=166) was a 3-bp indel localized in the first exon of MMGT1 (Membrane Magnesium Transporter 1), immediate 324-bp downstream of its transcription start site (TSS). Although the role of MMGT1 in prostate cancer has not been extensively characterized, by examining RNA-Seq data in TCGA prostate tumors, we observed significant down-regulation of MMGT1 across most of the samples of varying Gleason scores (FIG. 3B), supporting a significant role of MMGT1 in prostate cancer (see Discussion). Another extreme outlier is a somatic allele (a one-base substitution) in the intronic region of ARHGEF16 (Rho Guanine Nucleotide Exchange Factor 16), which was previously implicated in glioma etiology(40). We queried its expression in the TCGA prostate cancer cohort using UALCAN(23), and immediately observed its significant upregulation in prostate tumor samples relative to matched normal samples across all clinical grades (stratified by Gleason scores, FIG. 3C). Another extreme somatic allele (a one-base substitution) was identified in the promoter region of IPO11 (the Importin 11), indicating its strong effect on perturbing IPO11 promoter activity in the prostate. Interestingly, this gene has recently been identified as a tumor suppressor which prevents PTEN from degradation, and has been suggested as an indicator for clinical outcome of prostate cancer patients receiving radical prostatectomy(41). Querying TCGA expression data, we consistently observed its down-regulation in prostate tumor samples across varying Gleason score groups (FIG. 3D). Apparently, the detected somatic allele had contributed to prostate cancer etiology by dysregulating IPO11 expression in this patient. Notably all these extreme examples were specific to individual personal genomes, and did not form mutational hotspots in their neighboring regions across all samples. Therefore, these mutations would be missed in typical mutational recurrence analysis, but now can be effectively captured by our DEEP framework for personal genomes.

### Agnostically Identify Pathogenic Non-Coding Somatic Alleles in Prostate Cancer Genomes

In addition to these extreme outliers, we next set out to agnostically identify high-confidence pathogenic non-coding somatic alleles. Across the genome, we used the upper one percentile across all the 422,314 somatic mutations analyzed to identify the significant somatic mutations (receiving extreme DEEP scores), and detected 2,037 impactful somatic mutations in genic regions, including 48, 1914, 31, 3, and 41 in promoter, intronic, exonic, 5′UTR and 3′UTR regions, respectively. These significant genic mutations, together with 2,050 somatic mutations receiving significant DEEP scores (upper one percentile across the genome) in intergenic regions, characterized the altered epigenomes in localized prostate tumors.

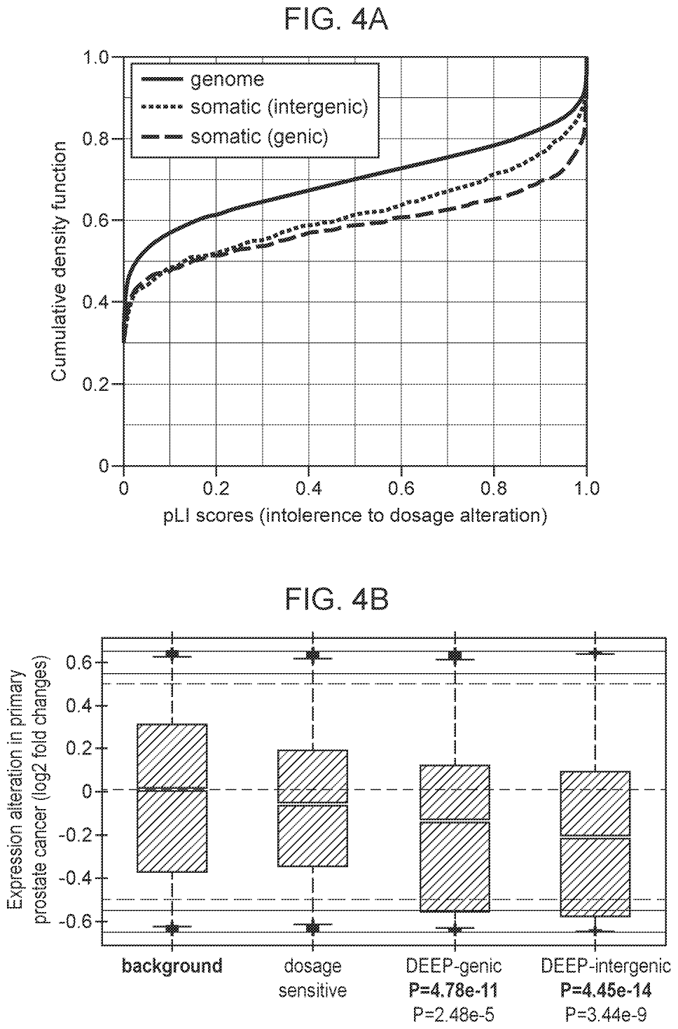

We reasoned that if those mutations were indeed implicated in prostate cancer pathogenesis, their associated genes are expected to be intolerant to expression alterations. We associated the mutations with their neighboring genes using the closest physical proximity, and compared the recently developed pLI scores for each gene, which have been widely used as a proxy of gene dosage sensitivity or haploinsufficiency(25). As expected, genes harboring significant genic significant non-coding somatic mutations indeed displayed substantially increased pLI scores relative to the genome background (P=1.98e-14, Wilcoxon rank-sum test, FIG. 4A), and the trend was also significant for genes physically associated with the significant intergenic mutations (P=4.77e-9, Wilcoxon rank-sum test, FIG. 4A). Taken together, we identified non-coding somatic mutations having extreme impacts on altering prostate epigenomic architecture, and their associated genes are more likely to be intolerant to expression alterations.

We followed previous practice and used pLI ≥0.9 to define dosage-sensitive genes(25). We identified 463 and 317 dosage-sensitive genes affected by significant genic and intergenic somatic mutations, respectively, and these mutations received extreme DEEP scores (DEEP-genic and DEEP-intergenic genes). To confirm their implication in primary prostate cancers, we examined RNA-Seq data in the TCGA-PRAD (prostate adenocarcinoma) cohort and computed expression fold changes for each gene in 497 primary prostate tumor samples relative to 55 normal prostate tissues. Referenced with genes harboring at least one somatic mutation, the DEEP-genic (P=4.78e-11, Wilcoxon rank-sum test, FIG. 4B) and DEEP-intergenic (P=4.45e-14, Wilcoxon rank-sum test, FIG. 4B) genes displayed substantial down-regulation. To exclude the possibility that the down-regulation was caused by their respective extreme pLI scores, we identified 3,230 genes with the same pLI threshold ≥0.9, and again, we observed that both DEEP-genic (P=2.48e-5, Wilcoxon rank-sum test) and DEEP-intergenic (P=3.44e-9, Wilcoxon rank-sum test) genes displayed significant downregulation relative to these dosage sensitive genes (FIG. 4B). Taken together, this comparison confirmed that our deep learning model indeed captured genes implicated in prostate cancer. Among these identified genes, Table 1 displayed 13 genes whose promoters were affected by significant somatic mutations. Ranking them based on our DEEP scores, we immediately observed that the top four genes, ING3, IPO11 (described above), LARP4 and TSC22D1, were all tumor suppressors(41-46), highlighting the strong and novel candidacies of these gene in prostate cancer. We particularly note that the top hit ING3 is necessary for ATM signaling and DNA repair for double-strand breaks(42). Next to the top four genes, MCL1 was ranked at the fifth position, which has been proposed as a target for cancer therapy(47-49). This list also included several other known prostate cancer genes such as HNRNPM(50) and NFKBIA(51), as well as CDK8, an emerging target for immunotherapy(52). A few other genes have uncharacterized functions in prostate cancer, and we confirmed their candidacy in prostate cancer based on their differential expression in the TCGA prostate tumor samples by querying UALCAN(23), including ZNF711(P=8.66e-13), DIDO1 (P=5.60e-3), LHX2 (P=1.17e-6) and MLLT3 (P=0.01). In addition to these genes with affected promoters, we also identified the androgen receptor AR with a significant intronic mutation receiving an extreme DEEP score, indicating perturbation on an intronic regulatory element in AR. Importantly, implementing ChIP Enrichment Analysis (ChEA/EnrichR)(53,54), we immediately observed that these genes (DEEP-genic and DEEP-intergenic) were highly enriched for targets of multiple transcription factors with the highest enrichment for AR (Table 2). This enrichment suggests that the affected genes were converged onto AR-mediated regulatory network. Moreover, referencing with the Human Proteome Map(27), both the DEEP-genic (P=2.95e-8, Wilcoxon rank-sum test) and DEEP-intergenic (P=0.02, Wilcoxon rank-sum test) genes displayed elevated protein abundance in the prostate gland, confirming their physiological functions. Taken together, these observations confirmed the implication of our identified non-coding somatic mutations in prostate cancer. Although these mutations were individually identified from personal genomes, they in fact convergent onto AR-mediated pathways in the prostate.

### The Perturbed AR Signaling Pathway

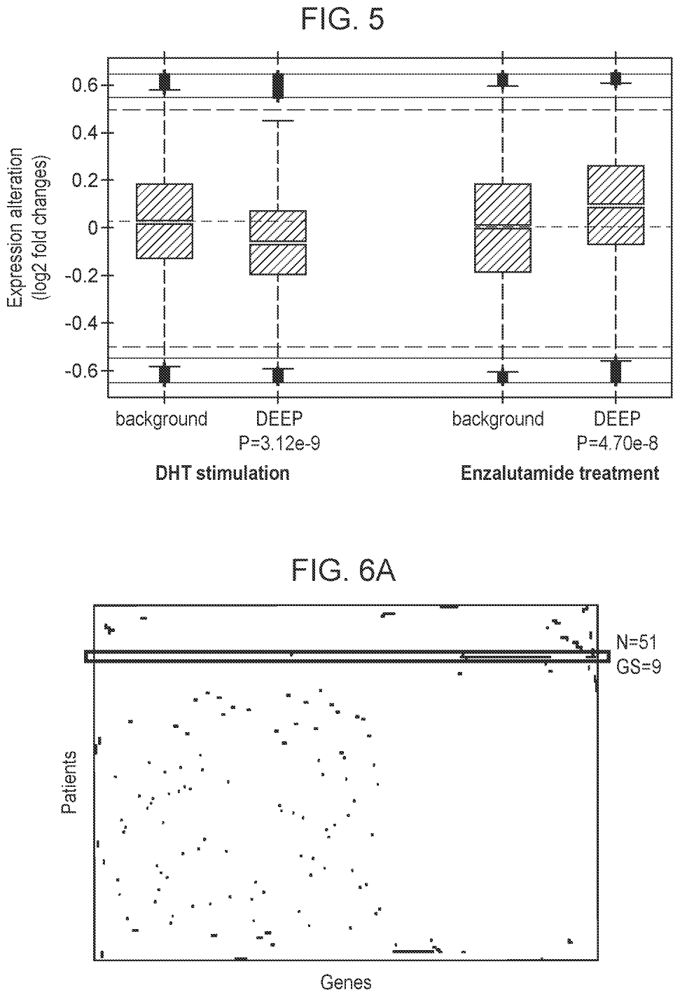

We next examined the identified genes for their roles in mediating AR signaling. We first considered DEEP-genic genes because these genes are less likely confounded by remote chromosomal interactions. We first considered the LNCaP prostate cancer cell line that was stimulated by an AR ligand, dihydrotestosterone (DHT, the physiological androgen), the AR ligand. We examined RNA-Seq data generated from a previous study(26), where transcriptome profiling was performed on LNCaP cells in 6 and 24 hours after DHT stimulation. Referencing with protein-coding genes harboring at least one genic somatic mutation, we observed that the identified 463 DEEP-genic genes displayed a significant down-regulation upon DHT stimulation (P=3.12e-9, Wilcoxon rank-sum test, FIG. 5) after 6 hours, whereas the expression alteration from 6 hours to 24 hours was insignificant (P=0.07, Wilcoxon rank-sum test).

We reasoned that if the downregulation suggests the implication of the identified genes in AR signaling pathways, we would expect their significant up-regulation when treating the LNCaP cells using an AR antagonist. From the same study(26), we further examined the RNA-Seq data for LNCaP after the enzalutamide treatment after 48 hours, using the 48-hour treatment of dimethyl sulfoxide (DMSO) as control. Enzalutamide is an approved and potent AR antagonist for treating castration-resistant prostate cancer, which achieves its antagonist function by switching DNA motifs recognized by AR(55). As expected, the identified genes indeed displayed significant up-regulation upon enzalutamide treatment (P=4.70e-8, Wilcoxon rank-sum test, FIG. 5), consistent with their marked down-regulation upon DHT simulation. We note that the observed expression alterations were also significant when compared with the list of 3,230 genes with the same pLI threshold at 0.9 (P<5e-5, Wilcoxon rank-sum test), excluding the possibility that these expression changes were merely explained by their dosage sensitivity. We also performed the same analysis on DEE P-intergenic genes, which displayed similar down-regulation and up-reregulation upon DHT simulation (P=1.64e-4, Wilcoxon rank-sum test) and enzalutamide treatment (P=5.02e-4, Wilcoxon rank-sum test), respectively. Taken together, the DHT stimulation and enzalutamide treatment mutually validated each other, and collectively demonstrated that our approach identified pathogenic mutations perturbing the AR signaling pathway in prostate cancer.

### Personal Genome Scan to Predict Clinical Outcomes

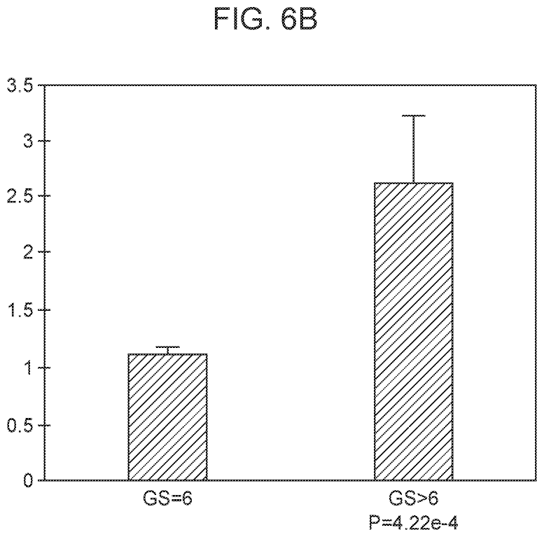

Given the mutational convergence onto AR signaling, we further hypothesized that at the personal genome level, accumulating pathogenic mutations affecting AR signaling are likely predictive of adverse clinical outcomes. We analyzed the distribution of the identified pathogenic regulatory somatic mutations in each personal cancer genome, followed by a comparison with their clinical records. We considered 302 individuals with available clinical information. Among the 463 DEEP-genic genes (deleterious somatic mutations falling in genic regions), we considered 257 that had at least one deleterious somatic mutation identified by DEEP in these patients and that were likely implicated in AR signaling given their up-regulation or down-regulation after DHT or enzalutamide treatment, respectively (FIG. 5). The mutation profile is shown in FIG. 6A, which immediately identified one extreme patient: 51 among the 257 genes were perturbed by somatic regulatory variations, suggesting a pervasive dysregulation of AR-mediated pathways. As expected, this extreme patient received a Gleason score of 9 (only two patients had the scores ≥9 in this cohort), suggesting a potential correlation between the number of affected genes and an adverse clinical outcome. To generalize this observation, we extended the analysis to the entire patient cohort, and identified 121 patients with available Gleason scores on clinical records. Strikingly, we observed a significant positive correlation between Gleason scores and the number of these genes affected in each personal cancer genome (Pearson's R=0.34, P=1.1e-4, Spearman's rho=0.33, P=2.38e-4). Stratifying patients based on their Gleason scores, individuals with milder tumors (Gleason score=6) on average had one affected gene per person, in sharp contrast to ˜2.5 among individuals with more aggressive tumors (Gleason score>6, P=4.22e-4, Wilcoxon rank-sum test, FIG. 6B). As an independent control experiment, we considered the somatic mutations receiving the lowest one percentile of DEEP scores across the genome in the same cohort (i.e. high-confidence benign variants, as opposed to the pathogenic mutations in the upper 1% in this cohort), and repeated the same analysis. However, no significant correlation could be observed (Pearson's R=0.06, P=0.4023, Spearman's rho=0.006, P=0.9232), demonstrating the specificity of our DEEP scoring system. In addition to those genic mutations (the DEEP-genic set), we also tested significant somatic regulatory mutations falling in intergenic regions, associating each mutation with their closest genes (the DEEP-intergenic set). We still observed similar positive correlations, albeit the statistical significance was weakened (Pearson's R=0.27, P=7.3e-3, Spearman's rho=0.18, P=0.07). This observation suggests potential remote chromosome interactions that could have complicated the assignment of intergenic mutations to their regulating target genes. Taken together, leveraging the panel of 257 genes, our approach could be deployed to estimate the expected Gleason scores by scanning each personal prostate cancer genome.

### The DEEP Platform for Analyzing Prostate Cancer Genomes.

Having established the effectiveness of using DEEP to capture deleterious mutations in prostate cancer genomes, we have made the software package available, which can be easily implemented in a standard UNIX environment. The system simply takes input of a VCF file, and will automatically compute the likelihood of disrupting prostate-specific chromatin architecture for each of the variants in the VCF file. The platform will prioritize high-confidence mutations using a set of criteria including pLI scores and TCGA gene expression as we performed in the analyses above. It is expected that the DEEP platform will significantly advance our understanding of the non-coding somatic mutations in the prostate cancer genome, and will help improve our clinical practice by providing a tool for personal genome scan.

### Discussion

Despite abundant somatic mutations in the non-coding genome, our current cancer genome analysis has been primarily focused on mutations in protein-coding sequences affecting ˜1.5% of the human genome. Analyzing recurrent somatic mutations has provided us a glimpse into the mechanisms of tumorigenesis by perturbing gene regulation(8); however, compared with the vast non-coding genome, somatic mutations are sparsely localized, and therefore inferring pathogenicity merely based on recurrent mutations or mutation hotspots could be less effective, leading to the observation of a paucity of non-coding driver mutations in cancer(8). However, in this study, the DEEP framework has aimed to directly assess allelic effects on altering chromatin architecture for each somatic mutation, thereby enabling us to identify pathogenic regulatory somatic mutations from personal cancer genomes. We applied this strategy to analyzing localized prostate cancer, and identified numerous novel pathogenic somatic alleles as well as their affected genes as novel candidate loci in prostate cancer. Our functional genomic analyses confirmed their function in prostate cancer, revealed the mutational convergence on AR-mediated pathways, and demonstrated the clinical utility of our approach for personal genome scans. With this new approach, we concluded that pathogenic regulatory somatic mutations are widely dispersed across the genome.

Applying DEEP to primary prostate tumor genomes, we examined the complete set of 422,314 somatic mutations from 306 patient tumor samples, and identified thousands of significant mutations receiving extreme DEEP scores (above one percentile) indicating their alteration of the prostate epigenome. Interestingly, genes affected by these identified somatic alleles are responsive to enzalutamide treatment, an FDA approved medication for treating prostate cancer(56,57). Therefore, it is reasonable to speculate that these mutation carriers likely had differential responses to enzalutamide than other patient groups. Future studies are therefore warranted to develop individualized therapeutic strategies utilizing personal somatic mutation profiles.

In addition to therapeutic strategies, we have also shown that our DEEP scoring scheme could be leveraged to assess clinical outcomes of prostate cancer given the significant correlation with patients' Gleason scores (FIG. 6). Therefore, compared with our current clinical practice to sequence cancer exomes/genomes to identify pathogenic missense and nonsense mutations, it is expected that our DEEP framework could be integrated in the clinical sequencing pipeline, extending our variant interpretation from coding sequences to the vast non-coding genome. We have made our software package available which can be readily used to scan prostate cancer genomes to better inform clinical practice.

We adopted a very conservative strategy to agnostically identify high-confidence somatic non-mutations in prostate cancer by only considering extreme DEEP and pLI scores. However, those not considered in our analysis could also strongly contribute to prostate cancer etiology. For example, MMGT1 (membrane magnesium transporter 1) received the highest DEEP score in our analysis (FIG. 3A), but its pLI score was 0.8 in the latest genomAD database(58). Although it was not included in our downstream functional genomic study adopting a stringent threshold pLI=0.9, this somatic mutation affecting MMGT1 is expected to contribute to prostate cancer, which was also supported by its down-regulation in the TGCA-PRAD cohort (FIG. 3B) across varying Gleason score groups, except for the most aggressive tumor group (Gleason score=10, small sample size). Intriguingly, previous work suggested that the competition between calcium and magnesium for membrane binding sites will result in an imbalanced cellular micronutrient in human diseases(59,60), and a higher calcium to magnesium dietary intake ratio is positively correlated with increased chance to develop aggressive prostate cancer(60,61). These observations are consistent with the perturbed gene regulation of MMGT1 by a somatic mutation in this study, and are also supported by the reduced MMGT1 expression in the TCGA (primary prostate cancer) cohort: by reducing the abundance of membrane magnesium transporter, the magnesium intake would be decreased accordingly, leading to an increased calcium to magnesium ratio and therefore an increased chance to develop aggressive prostate cancer. This finding also highlights the possibility of personalizing the dietary plan to manage prostate cancer based on individuals' genomic profiles. Taken together, for variants receiving intermediate DEEP scores, they also likely contribute to prostate cancer, and their implication requires further investigation based on our clinical expertise and understanding of gene-environment interaction (e.g. patient lifestyles). Overall, our DEEP framework provided a comprehensive resource to re-annotate the prostate cancer genome.

We observed that many ATAC-Seq peaks in the prostate gland are localized in exonic regions, suggesting potential enhancer elements for nearby genes. We also identified several somatic mutations that likely alter chromatin structure in exonic regions. Therefore, when analyzing coding sequencing mutations, extra caution has to be executed to determine the mutational effect on gene regulation. This notion was also suggested in previous study(62,63). In a similar vein, numerous intronic and intergenic regulatory elements were also observed in the prostate and impactful mutations residing these elements were also identified using our DEEP framework. To gain further mechanistic insights, extensive epigenome profiling experiments have to be performed to systematically characterize the epigenome landscape in the prostate, such as ChIP-Seq profiling for enhancer (H3K4me1 and H3K27ac) and promoter (H3K4me3) marks. The DEEP framework can be further expanded to incorporate these epigenome data, which will help us determine the perturbed regulatory network in prostate cancer.

On the methodological side, our DEEP framework is purely driven by the allelic effects on altering chromatin structure without requiring an aggregation of large-scale clinical samples. We compared the DEEP scores with evolutionary conservation (GERP++ scores)(29), and observed that their correlation was close to 0, which in line with a previous work suggesting that genetic loci implicated in prostate cancer (appearing in later stages of one's life) are selectively neutral(64). The lack of substantial correlation was also observed when comparing the DEEP scores with CADD scores(65). This is expected because much contribution to CADD scoring comes from evolutionary conservation. CADD also incorporates epigenome information, which however only examines the localization of mutations in regulatory elements without quantifying the allelic effects(65). More importantly, CADD aggregates all existing epigenome information without considering tissue specificity. However, we specifically trained our model using the prostate epigenome, so the mutational pathogenicity is solely defined in the prostate-specific context. Given the lack of correlation between our algorithm with CADD and GERP++, apparently the information we captured in our analysis would be missed by the other approaches. Because of the flexible design (only requiring tissue-specific epigenomes), DEEP is readily extended to studying any other cancer types, and can be easily deployed as a clinical tool for prioritizing clinically relevant mutations in the non-coding genome.

### REFERENCES

1. International Cancer Genome, C., Hudson, T. J., Anderson, W., Artez, A., Barker, A. D., Bell, C., Bernabe, R. R., Bhan, M. K., Calvo, F., Eerola, I. et al. (2010) International network of cancer genome projects. Nature, 464, 993-998.
2. Corradin, O. and Scacheri, P. C. (2014) Enhancer variants: evaluating functions in common disease. Genome Med, 6, 85.
3. Schaub, M. A., Boyle, A. P., Kundaje, A., Batzoglou, S. and Snyder, M. (2012) Linking disease associations with regulatory information in the human genome. Genome Res, 22, 1748-1759.
4. Khurana, E., Fu, Y., Chakravarty, D., Demichelis, F., Rubin, M. A. and Gerstein, M. (2016) Role of non-coding sequence variants in cancer. Nat Rev Genet, 17, 93-108.
5. Huang, F. W., Hodis, E., Xu, M. J., Kryukov, G. V., Chin, L. and Garraway, L. A. (2013) Highly recurrent TERT promoter mutations in human melanoma. Science, 339, 957-959.
6. Zhou, S., Hawley, J. R., Soares, F., Grillo, G., Teng, M., Madani Tonekaboni, S. A., Hua, J. T., Kron, K. J., Mazrooei, P., Ahmed, M. et al. (2020) Noncoding mutations target cis-regulatory elements of the FOXA1 plexus in prostate cancer. Nat Commun, 11, 441.
7. Corces, M. R., Granja, J. M., Shams, S., Louie, B. H., Seoane, J. A., Zhou, W., Silva, T. C., Groeneveld, C., Wong, C. K., Cho, S. W. et al. (2018) The chromatin accessibility landscape of primary human cancers. Science, 362.
8. Rheinbay, E., Nielsen, M. M., Abascal, F., Wala, J. A., Shapira, O., Tiao, G., Hornshoj, H., Hess, J. M., Juul, R. I., Lin, Z. et al. (2020) Analyses of non-coding somatic drivers in 2,658 cancer whole genomes. Nature, 578, 102-111.
9. Gan, K. A., Carrasco Pro, S., Sewell, J. A. and Fuxman Bass, J. I. (2018) Identification of Single Nucleotide Non-coding Driver Mutations in Cancer. Front Genet, 9, 16.
10. Piraino, S. W. and Furney, S. J. (2016) Beyond the exome: the role of non-coding somatic mutations in cancer. Ann Oncol, 27, 240-248.
11. Zhang, C., Xuan, Z., Otto, S., Hover, J. R., McCorkle, S. R., Mandel, G. and Zhang, M. Q. (2006) A clustering property of highly-degenerate transcription factor binding sites in the mammalian genome. Nucleic Acids Res, 34, 2238-2246.
12. Slattery, M., Zhou, T., Yang, L., Dantas Machado, A. C., Gordan, R. and Rohs, R. (2014) Absence of a simple code: how transcription factors read the genome. Trends Biochem Sci, 39, 381-399.
13. Cheng, Z. e.a. (2020) A catalog of cis-regulatory mutations in 12 major cancer types. bioRxiv.
14. Zhang, W., Bojorquez-Gomez, A., Velez, D. O., Xu, G., Sanchez, K. S., Shen, J. P., Chen, K., Licon, K., Melton, C., Olson, K. M. et al. (2018) A global transcriptional network connecting noncoding mutations to changes in tumor gene expression. Nat Genet, 50, 613-620.
15. Heyn, H. (2016) Quantitative Trait Loci Identify Functional Noncoding Variation in Cancer. PLoS Genet, 12, e1005826.
16. Espiritu, S. M. G., Liu, L. Y., Rubanova, Y., Bhandari, V., Holgersen, E. M., Szyca, L. M., Fox, N. S., Chua, M. L. K., Yamaguchi, T. N., Heisler, L. E. et al. (2018) The Evolutionary Landscape of Localized Prostate Cancers Drives Clinical Aggression. Cell, 173, 1003-1013 e1015.
17. Fraser, M., Sabelnykova, V. Y., Yamaguchi, T. N., Heisler, L. E., Livingstone, J., Huang, V., Shiah, Y. J., Yousif, F., Lin, X., Masella, A. P. et al. (2017) Genomic hallmarks of localized, non-indolent prostate cancer. Nature, 541, 359-364.
18. Taplin, M. E. (2007) Drug insight: role of the androgen receptor in the development and progression of prostate cancer. Nat Clin Pract Oncol, 4, 236-244.
19. Matsumoto, T., Sakari, M., Okada, M., Yokoyama, A., Takahashi, S., Kouzmenko, A. and Kato, S. (2013) The androgen receptor in health and disease. Annu Rev Physiol, 75, 201-224.
20. Heinz, S., Benner, C., Spann, N., Bertolino, E., Lin, Y. C., Laslo, P., Cheng, J. X., Murre, C., Singh, H. and Glass, C. K. (2010) Simple combinations of lineage-determining transcription factors prime cis-regulatory elements required for macrophage and B cell identities. Mol Cell, 38, 576-589.
21. Consortium, E. P. (2012) An integrated encyclopedia of DNA elements in the human genome. Nature, 489, 57-74.
22. Tarbell, E. D. and Liu, T. (2019) HMMRATAC: a Hidden Markov ModeleR for ATAC-seq. Nucleic Acids Res, 47, e91.
23. Chandrashekar, D. S., Bashel, B., Balasubramanya, S. A. H., Creighton, C. J., Ponce-Rodriguez, I., Chakravarthi, B. and Varambally, S. (2017) UALCAN: A Portal for Facilitating Tumor Subgroup Gene Expression and Survival Analyses. Neoplasia, 19, 649-658.
24. Love, M. I., Huber, W. and Anders, S. (2014) Moderated estimation of fold change and dispersion for RNA-seq data with DESeq2. Genome Biol, 15, 550.
25. Lek, M., Karczewski, K. J., Minikel, E. V., Samocha, K. E., Banks, E., Fennell, T., O'Donnell-Luria, A. H., Ware, J. S., Hill, A. J., Cummings, B. B. et al. (2016) Analysis of protein-coding genetic variation in 60,706 humans. Nature, 536, 285-291.
26. Zhang, Y., Pitchiaya, S., Cieslik, M., Niknafs, Y. S., Tien, J. C., Hosono, Y., Iyer, M. K., Yazdani, S., Subramaniam, S., Shukla, S. K. et al. (2018) Analysis of the androgen receptor-regulated lncRNA landscape identifies a role for ARLNC1 in prostate cancer progression. Nat Genet, 50, 814-824.
27. Kim, M. S., Pinto, S. M., Getnet, D., Nirujogi, R. S., Manda, S. S., Chaerkady, R., Madugundu, A. K., Kelkar, D. S., Isserlin, R., Jain, S. et al. (2014) A draft map of the human proteome. Nature, 509, 575-581.
28. Rentzsch, P., Witten, D., Cooper, G. M., Shendure, J. and Kircher, M. (2019) CADD: predicting the deleteriousness of variants throughout the human genome. Nucleic Acids Res, 47, D886-D894.
29. Davydov, E. V., Goode, D. L., Sirota, M., Cooper, G. M., Sidow, A. and Batzoglou, S. (2010) Identifying a high fraction of the human genome to be under selective constraint using GERP++. PLoS Comput Biol, 6, e1001025.
30. Zhou, J. and Troyanskaya, O. G. (2015) Predicting effects of noncoding variants with deep learning-based sequence model. Nat Methods, 12, 931-934.
31. Zhou, J., Theesfeld, C. L., Yao, K., Chen, K. M., Wong, A. K. and Troyanskaya, O. G. (2018) Deep learning sequence-based ab initio prediction of variant effects on expression and disease risk. Nat Genet, 50, 1171-1179.
32. Buenrostro, J. D., Wu, B., Chang, H. Y. and Greenleaf, W. J. (2015) ATAC-seq:

A Method for Assaying Chromatin Accessibility Genome-Wide. Curr Protoc Mol Biol, 109, 21 29 21-21 29 29.
33. Bhatia-Gaur, R., Donjacour, A. A., Sciavolino, P. J., Kim, M., Desai, N., Young, P., Norton, C. R., Gridley, T., Cardiff, R. D., Cunha, G. R. et al. (1999) Roles for Nkx3.1 in prostate development and cancer. Genes Dev, 13, 966-977.
34. Bowen, C., Bubendorf, L., Voeller, H. J., Slack, R., Willi, N., Sauter, G., Gasser, T. C., Koivisto, P., Lack, E. E., Kononen, J. et al. (2000) Loss of NKX3.1 expression in human prostate cancers correlates with tumor progression. Cancer Res, 60, 6111-6115.
35. Ecke, T. H., Schlechte, H. H., Schiemenz, K., Sachs, M. D., Lenk, S. V., Rudolph, B. D. and Loening, S. A. (2010) TP53 gene mutations in prostate cancer progression. Anticancer Res, 30, 1579-1586.
36. Lee, D., Corkin, D. U., Baker, M., Strober, B. J., Asoni, A. L., McCallion, A. S. and Beer, M. A. (2015) A method to predict the impact of regulatory variants from DNA sequence. Nat Genet, 47, 955-961.
37. Shrikumar, A., Prakash, E. and Kundaje, A. (2019) GkmExplain: fast and accurate interpretation of nonlinear gapped k-mer SVMs. Bioinformatics, 35, i173-i182.
38. Zhou, J., Park, C. Y., Theesfeld, C. L., Wong, A. K., Yuan, Y., Scheckel, C., Fak, J. J., Funk, J., Yao, K., Tajima, Y. et al. (2019) Whole-genome deep-learning analysis identifies contribution of noncoding mutations to autism risk. Nat Genet, 51, 973-980.
39. Roadmap Epigenomics, C., Kundaje, A., Meuleman, W., Ernst, J., Bilenky, M., Yen, A., Heravi-Moussavi, A., Kheradpour, P., Zhang, Z., Wang, J. et al. (2015) Integrative analysis of 111 reference human epigenomes. Nature, 518, 317-330.
40. Huang, D., Wang, Y., Xu, L., Chen, L., Cheng, M., Shi, W., Xiong, H., Zalli, D. and Luo, S. (2018) GLI2 promotes cell proliferation and migration through transcriptional activation of ARHGEF16 in human glioma cells. J Exp Clin Cancer Res, 37, 247.
41. Chen, M., Nowak, D. G., Narula, N., Robinson, B., Watrud, K., Ambrico, A., Herzka, T. M., Zeeman, M. E., Minderer, M., Zheng, W. et al. (2017) The nuclear transport receptor Importin-11 is a tumor suppressor that maintains PTEN protein. J Cell Biol, 216, 641-656.
42. Mouche, A., Archambeau, J., Ricordel, C., Chaillot, L., Bigot, N., Guillaudeux, T., Grenon, M. and Pedeux, R. (2019) ING3 is required for ATM signaling and DNA repair in response to DNA double strand breaks. Cell Death Differ, 26, 2344-2357.
43. Egiz, M., Usui, T., Ishibashi, M., Zhang, X., Shigeta, S., Toyoshima, M., Kitatani, K. and Yaegashi, N. (2019) La-Related Protein 4 as a Suppressor for Motility of Ovarian Cancer Cells. Tohoku J Exp Med, 247, 59-67.
44. Seetharaman, S., Flemyng, E., Shen, J., Conte, M. R. and Ridley, A. J. (2016) The RNA-binding protein LARP4 regulates cancer cell migration and invasion. Cytoskeleton (Hoboken), 73, 680-690.
45. Nakashiro, K., Kawamata, H., Hino, S., Uchida, D., Miwa, Y., Hamano, H., Omotehara, F., Yoshida, H. and Sato, M. (1998) Down-regulation of TSC-22 (transforming growth factor beta-stimulated clone 22) markedly enhances the growth of a human salivary gland cancer cell line in vitro and in vivo. Cancer Res, 58, 549-555.
46. Rentsch, C. A., Cecchini, M. G., Schwaninger, R., Germann, M., Markwalder, R., Heller, M., van der Pluijm, G., Thalmann, G. N. and Wetterwald, A. (2006) Differential expression of TGFbeta-stimulated clone 22 in normal prostate and prostate cancer. Int J Cancer, 118, 899-906.
47. Arai, S., Jonas, O., Whitman, M. A., Corey, E., Balk, S. P. and Chen, S. (2018) Tyrosine Kinase Inhibitors Increase MCL1 Degradation and in Combination with BCLXL/BCL2 Inhibitors Drive Prostate Cancer Apoptosis. Clin Cancer Res, 24, 5458-5470.
48. Merino, D., Kelly, G. L., Lessene, G., Wei, A. H., Roberts, A. W. and Strasser, A. (2018) BH3-Mimetic Drugs: Blazing the Trail for New Cancer Medicines. Cancer Cell, 34, 879-891.
49. Senichkin, V. V., Streletskaia, A. Y., Zhivotovsky, B. and Kopeina, G. S. (2019) Molecular Comprehension of Mcl-1: From Gene Structure to Cancer Therapy. Trends Cell Biol, 29, 549-562.
50. Yang, T., An, Z., Zhang, C., Wang, Z., Wang, X., Liu, Y., Du, E., Liu, R., Zhang, Z. and Xu, Y. (2019) hnRNPM, a potential mediator of YY1 in promoting the epithelial-mesenchymal transition of prostate cancer cells. Prostate, 79, 1199-1210.
51. Carter, S. L., Centenera, M. M., Tilley, W. D., Selth, L. A. and Butler, L. M. (2016) IkappaBalpha mediates prostate cancer cell death induced by combinatorial targeting of the androgen receptor. BMC Cancer, 16, 141.
52. Philip, S., Kumarasiri, M., Teo, T., Yu, M. and Wang, S. (2018) Cyclin-Dependent Kinase 8: A New Hope in Targeted Cancer Therapy? J Med Chem, 61, 5073-5092.
53. Lachmann, A., Xu, H., Krishnan, J., Berger, S. I., Mazloom, A. R. and Ma'ayan, A. (2010) ChEA: transcription factor regulation inferred from integrating genome-wide ChIP-X experiments. Bioinformatics, 26, 2438-2444.
54. Chen, E. Y., Tan, C. M., Kou, Y., Duan, Q., Wang, Z., Meirelles, G. V., Clark, N. R. and Ma'ayan, A. (2013) Enrichr: interactive and collaborative HTML5 gene list enrichment analysis tool. BMC Bioinformatics, 14, 128.
55. Chen, Z., Lan, X., Thomas-Ahner, J. M., Wu, D., Liu, X., Ye, Z., Wang, L., Sunkel, B., Grenade, C., Chen, J. et al. (2015) Agonist and antagonist switch DNA motifs recognized by human androgen receptor in prostate cancer. EMBO J, 34, 502-516.
56. Hussain, M., Fizazi, K., Saad, F., Rathenborg, P., Shore, N., Ferreira, U., Ivashchenko, P., Demirhan, E., Modelska, K., Phung et al. (2018) Enzalutamide in Men with Nonmetastatic, Castration-Resistant Prostate Cancer. N Engl J Med, 378, 2465-2474.
57. Beer, T. M., Armstrong, A. J., Rathkopf, D., Loriot, Y., Sternberg, C. N., Higano, C. S., Iversen, P., Evans, C. P., Kim, C. S., Kimura, G. et al. (2017) Enzalutamide in Men with Chemotherapy-naive Metastatic Castration-resistant Prostate Cancer: Extended Analysis of the Phase 3 PREVAIL Study. Eur Urol, 71, 151-154.
58. Karczewski, K. J. e.a. (2020) The mutational constraint spectrum quantified from variation in 141,456 humans. bioRxiv.
59. Rosanoff, A., Dai, Q. and Shapses, S. A. (2016) Essential Nutrient Interactions: Does Low or Suboptimal Magnesium Status Interact with Vitamin D and/or Calcium Status? Adv Nutr, 7, 25-43.
60. Dai, Q., Motley, S. S., Smith, J. A., Jr., Concepcion, R., Barocas, D., Byerly, S. and Fowke, J. H. (2011) Blood magnesium, and the interaction with calcium, on the risk of high-grade prostate cancer. PLoS One, 6, e18237.
61. Steck, S. E., Omofuma, O. O., Su, L. J., Maise, A. A., Woloszynska-Read, A., Johnson, C. S., Zhang, H., Bensen, J. T., Fontham, E. T. H., Mohler, J. L. et al. (2018) Calcium, magnesium, and whole-milk intakes and high-aggressive prostate cancer in the North Carolina-Louisiana Prostate Cancer Project (PCaP). Am J Clin Nutr, 107, 799-807.
62. Ahituv, N. (2016) Exonic enhancers: proceed with caution in exome and genome sequencing studies. Genome Med, 8, 14.
63. Birnbaum, R. Y., Clowney, E. J., Agamy, O., Kim, M. J., Zhao, J., Yamanaka, T., Pappalardo, Z., Clarke, S. L., Wenger, A. M., Nguyen, L. et al. (2012) Coding exons function as tissue-specific enhancers of nearby genes. Genome Res, 22, 1059-1068.
64. Lachance, J., Berens, A. J., Hansen, M. E. B., Teng, A. K., Tishkoff, S. A. and Rebbeck, T. R. (2018) Genetic Hitchhiking and Population Bottlenecks Contribute to Prostate Cancer Disparities in Men of African Descent. Cancer Res, 78, 2432-2443.
65. Kircher, M., Witten, D. M., Jain, P., O'Roak, B. J., Cooper, G. M. and Shendure, J. (2014) A general framework for estimating the relative pathogenicity of human genetic variants. Nat Genet, 46, 310-315.

TABLE 1
Significant non-coding somatic mutations in gene promoter regions.

| | | | | | DEEP | dist. to | | | TCGA-PRAD |
|---|---|---|---|---|---|---|---|---|---|
| chr | start | end | ref | alt | score | TSS | symbol | pLI | misexpression |
| chr7 | 1.21E+08 | 120590816 | C | T | 62.09 | −15 | ING3 | 0.99360296 | yes |
| chr5 | 61714006 | 61714006 | C | T | 53.89 | −705 | IPO11 | 0.99645696 | yes |
| chr12 | 50795199 | 50795199 | C | G | 33.42 | 9 | LARP4 | 0.99462886 | no |
| chr13 | 45151140 | 45151140 | C | G | 33.20 | −439 | TSC22D1 | 0.94115155 | yes |
| chr1 | 1.51E+08 | 150552163 | A | C | 27.69 | 51 | MCL1 | 0.95009297 | no |
| chrX | 1.2E+08 | 119709898 | C | T | 25.30 | −214 | CUL4B | 0.99984668 | no |
| chrX | 84499744 | 84499744 | G | A | 22.38 | 747 | ZNF711 | 0.98939356 | yes |
| chr20 | 61569348 | 61569348 | C | A | 22.30 | −44 | DIDO1 | 0.99999077 | yes |
| chr13 | 26827888 | 26827888 | T | A | 21.80 | −378 | CDK8 | 0.94665335 | yes |
| chr19 | 8509668 | 8509668 | G | A | 20.20 | −191 | HNRNPM | 0.99994805 | yes |
| chr9 | 1.27E+08 | 126773773 | C | A | 17.86 | −274 | LHX2 | 0.94678969 | yes |
| chr14 | 35873926 | 35873926 | G | A | 16.71 | 34 | NFKBIA | 0.97935203 | yes |
| chr9 | 20622011 | 20622011 | G | C | 15.55 | −74 | MLLT3 | 0.99559105 | yes |

TABLE 2
Enrichment of genes targeted by transcription
factors (TF). The TF that received the highest
enrichment was the androgen receptor (AR).

| | | | Adjusted | |
|---|---|---|---|---|
| TFs | Overlap | P-value | P-value | Odds Ratio |
| AR | 87/1095 | 1.50E−24 | 1.56E−22 | 3.4320542 |
| SMAD4 | 61/584 | 3.25E−23 | 1.69E−21 | 4.51196781 |
| REST | 84/1280 | 2.19E−18 | 7.59E−17 | 2.83477322 |
| SUZ12 | 90/1684 | 3.65E−14 | 9.48E−13 | 2.30860391 |
| NFE2L2 | 65/1022 | 1.14E−13 | 2.36E−12 | 2.74733403 |
| UBTF | 86/1631 | 3.24E−13 | 5.62E−12 | 2.27768412 |
| GATA1 | 49/807 | 7.71E−10 | 1.15E−08 | 2.6228385 |
| TCF3 | 50/1006 | 3.15E−07 | 4.09E−06 | 2.14694554 |
| SOX2 | 39/775 | 5.04E−06 | 5.83E−05 | 2.17376158 |
| STAT3 | 14/177 | 6.75E−05 | 7.02E−04 | 3.41667582 |
| GATA2 | 35/772 | 1.28E−04 | 0.00120975 | 1.95839255 |
| TCF3 | 37/840 | 1.47E−04 | 0.00127212 | 1.90270493 |
| PBX3 | 50/1269 | 1.66E−04 | 0.00132963 | 1.7019915 |
| REST | 21/383 | 2.55E−04 | 0.00189647 | 2.36847893 |
| RUNX1 | 50/1294 | 2.63E−04 | 0.00182612 | 1.66910913 |
| CHD1 | 29/655 | 7.00E−04 | 0.00455058 | 1.91251875 |
| CREB1 | 52/1444 | 0.00101017 | 0.00617986 | 1.55555423 |
| EZH2 | 13/237 | 0.00366662 | 0.0211849 | 2.3694307 |
| RCOR1 | 28/702 | 0.00387253 | 0.02119702 | 1.72293909 |
| SALL4 | 17/355 | 0.00397756 | 0.0206833 | 2.06856691 |
| TAF1 | 97/3346 | 0.00958424 | 0.04746483 | 1.25226085 |

### Example 2

### DEEP Plus Model

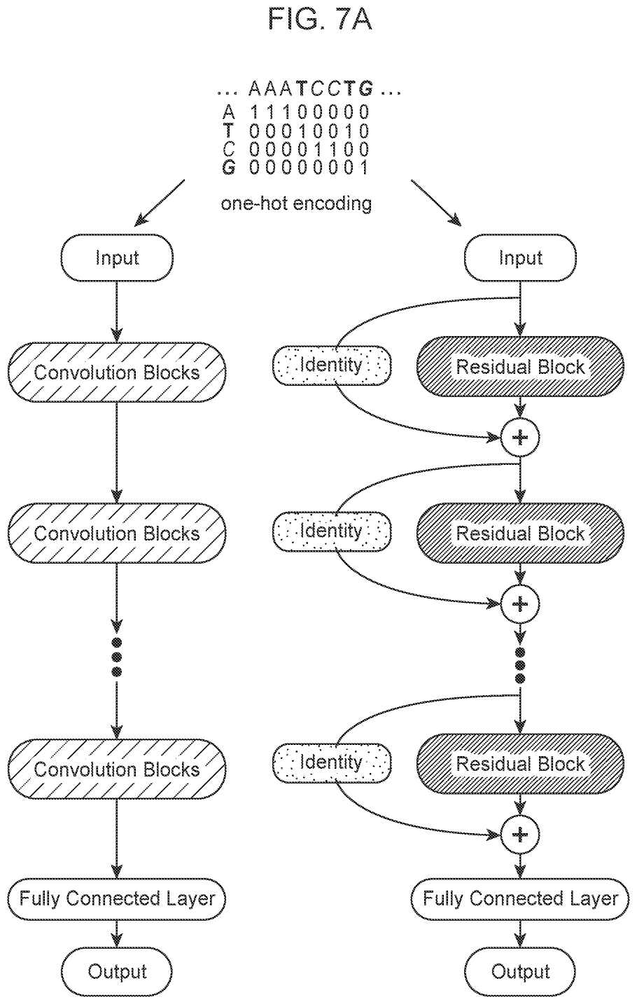

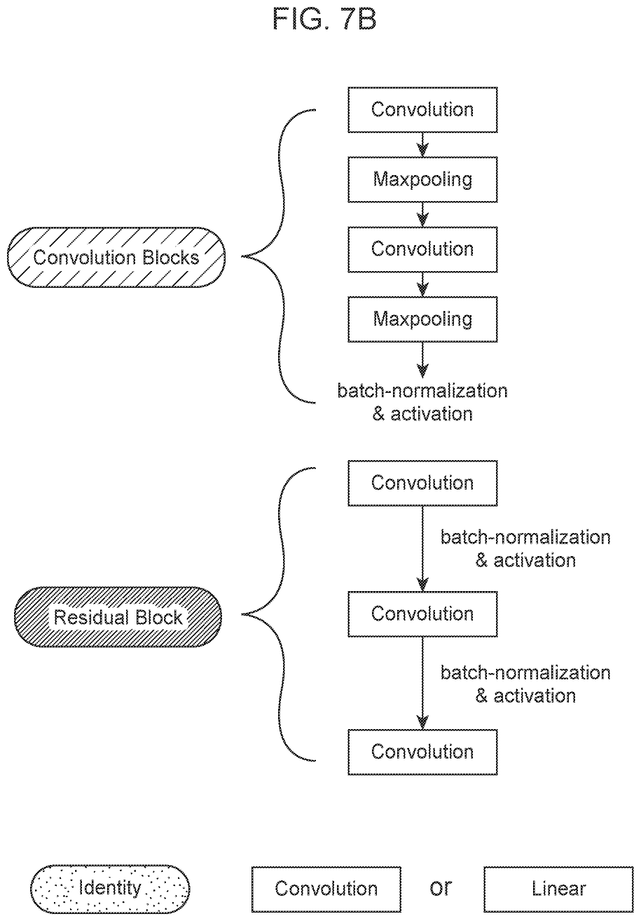

We devised an advanced version of our DEEP model, DEEP plus (DEEP+), based on the concepts of deep residual neural network. We substituted the building units in our DEEP framework with residual blocks that integrate the information adapted from both current and previous layers (FIG. 7). The reconstituted DEEP+ framework is easier for optimization during model training and expand the accuracy upon multilayer accumulation comparing to the previous version. It is conceivable that features extracted from genomic context are not always at the same scale, thereby resulting that the increment on model performance would be saturated or even drop dramatically when convolution neural network (CNN)-based DEEP model goes deeper. The suboptimal performance was often caused by the vanishing or exploding gradient in deep layers rather than overfitting when modeling complexity within the genome context. Our DEEP+ framework utilized the identity shortcuts that connect the hidden layers upper to circumvent this issue. The identity functions are learned to represent the residual information from upper layers so that unparalleled scaled features extracted from the genome could be better integrated.

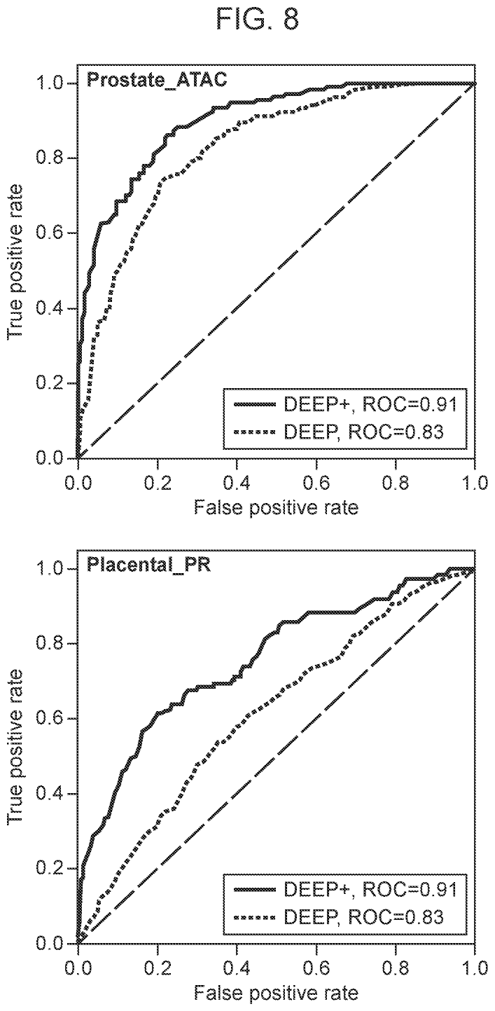

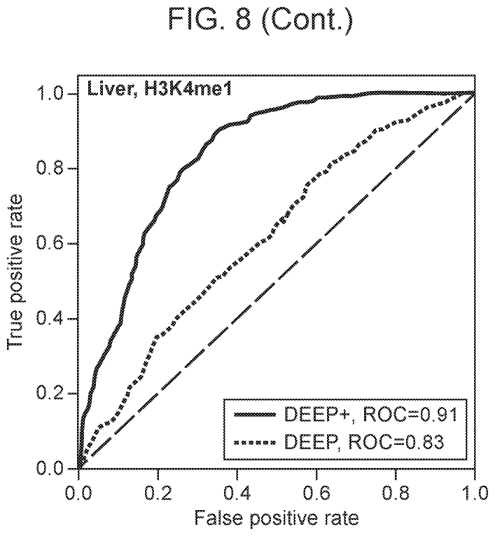

Based on such concepts, we adapted data with different genome context including ATAC-seq, histone ChIP and transcription factor ChIP from different human tissues as the input for our DEEP+ training. After we trained both DEEP+ and DEEP models with the same iterations and validation approaches, we observed that DEEP+ demonstrated higher predicting performance on the same test sets at different chromatin accessibility scales. (FIG. 8, indicated by AUROC) DEEP+ model not only increases the accuracy of high confident chromatin structure prediction (e.g., ATAC-seq in prostate gland), but also expands the capacity of predicting chromatin structures of higher complexity and less organization (e.g. ChIP-seq of PR in placenta and H3K4me1 in liver). Hence, we proposed that our DEEP+ model that incorporated residual deep learning framework could be more robust and ideal for capturing complex genome context.

What is claimed is:

1. A method for genome-wide identification of pathogenic non-coding somatic mutations associated with cancer, the method comprising:
a) providing a database comprising cancer-specific epigenomic correlation data for associations between non-coding somatic mutations and tissue-specific chromatin structural changes associated with tumorigenesis and cancer progression based on genome-wide epigenomic screening of a population of cancer patients;
b) generating a deep learning model to compute the probability that a given cancer genomic sequence has an open chromatin structure; and
c) using the deep learning model to identify pathogenic non-coding somatic mutations in a cancer genome, wherein a non-coding somatic mutation is considered to be pathogenic if an allelic change from its corresponding reference wild-type allele to the somatic mutation results in an alteration in predicted chromatin openness based on the deep learning model.

2. The method of claim 1, wherein the deep learning model uses a convolutional neural network or a deep residual neural network.

3. The method of claim 2, further comprising calculating a deep estimation from epigenome prediction (DEEP) score or a DEEP+ score for each non-coding somatic mutation that is identified as pathogenic.

4. The method of any one of claims 1 to 3, wherein the cancer is prostate cancer.

5. The method of any one of claims 1 to 4, wherein the cancer is non-metastatic.

6. The method of any one of claims 1 to 5, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

7. The method of any one of claims 1 to 6, wherein the non-coding somatic mutations comprise at least one insertion, deletion, or single-nucleotide variant.

8. The method of any one of claims 1 to 7, wherein the epigenomic correlation data comprises assay for transposase-accessible chromatin sequencing (ATAC-Seq) data.

9. A method of predicting risk of tumorigenesis or cancer progression in an individual, the method comprising:
a) obtaining a biological sample suspected of comprising cancerous or premalignant cells from the individual;
b) genotyping one or more cells in the biological sample to determine if the individual has one or more pathogenic non-coding somatic mutations; and
c) calculating a composite deep estimation from epigenome prediction (DEEP) score or a DEEP+ score for the pathogenic non-coding somatic mutations detected by genotyping, wherein the composite DEEP score or DEEP+ score indicates the risk of tumorigenesis or cancer progression in the individual.

10. The method of claim 9, wherein the non-coding somatic mutations are in an intronic genomic region, a promoter, a 5′ untranslated region (5′ UTR), a 3′ untranslated region (3′ UTR), an exonic genomic region, an intergenic genomic region, or a genomic region encoding a non-coding RNA.

11. The method of claim 9 or 10, wherein the non-coding somatic mutations comprise at least one insertion, deletion, or single-nucleotide variant.

12. The method of any one of claims 9 to 11, wherein the cancer is non-metastatic.

13. The method of any one of claims 9 to 12, wherein the cancer is prostate cancer.

14. The method of claim 13, wherein the one or more pathogenic non-coding somatic mutations comprise one or more pathogenic non-coding somatic mutations selected from Table 1.

15. The method of any one of claims 9 to 14, further comprising predicting responsiveness of the individual to treatment with an androgen receptor inhibitor based on identifying one or more pathogenic non-coding somatic mutations that alter regulation of a gene responsive to 5α-dihydrotestosterone (DHT).

16. The method of any one of claims 9 to 15, wherein the one or more pathogenic non-coding somatic mutations comprise at least one pathogenic non-coding somatic mutation in a gene selected from the group consisting of ING3, IPO11, LARP4, TSC22D1, MCL1, CUL4B, ZNF711, DIDO1, CDK8, HNRNPM, LHX2, NFKBIA, and MLLT3.

17. The method of any one of claims 9 to 16, further comprising calculating a composite pLI score for the pathogenic non-coding somatic mutations detected in the individual by genotyping, wherein the composite DEEP score or DEEP+ score is used in combination with the composite pLI score to determine the risk of prostate cancer progression in the individual.

18. The method of any one of claims 9 to 17, wherein said genotyping comprises sequencing at least part of a genome of the one or more cancerous cells from the biological sample.

19. The method of claim 18, wherein said genotyping comprises sequencing the whole genome of the one or more cells from the biological sample.

20. The method of any one of claims 9 to 19, wherein the biological sample is a tumor biopsy, a tumor surgical specimen, or blood comprising circulating tumor cells.

21. The method of any one of claims 9 to 20, further comprising performing medical imaging of a site of interest in the individual that is suspected of being cancerous, for example, by magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, or fluorescence imaging.

22. The method of any one of claims 9 to 21, further comprising treating the individual for the cancer if the composite DEEP score or DEEP+ score indicates the individual is at risk of cancer progression.

23. The method of claim 22, wherein said treating comprises surgery, radiation therapy, chemotherapy, hormonal therapy, immunotherapy, anti-angiogenic therapy, molecularly targeted or biologic therapy, or photodynamic therapy, or a combination thereof.

24. A database comprising DEEP scores or DEEP+ scores for a plurality of pathogenic non-coding somatic mutations associated with tumorigenesis or cancer progression, wherein the DEEP scores or DEEP+ scores are calculated according to the method of any one of claims 1 to 8.

25. The database of claim 24, wherein the database comprises or consists of Deep scores or DEEP+ scores for pathogenic non-coding somatic mutations selected from Table 1.

26. A computer implemented method for predicting risk of prostate cancer progression in an individual, the computer performing steps comprising:
a) receiving prostate cancer genome sequencing data for an individual;
b) identifying pathogenic non-coding somatic mutations present in the individual from the prostate cancer genome sequencing data, wherein the individual has a plurality of pathogenic non-coding somatic mutations selected from Table 1;
c) calculating a composite deep estimation from epigenome prediction (DEEP) score or a DEEP+ score for the pathogenic non-coding somatic mutations detected in the individual by genotyping using the database of claim 25, wherein the composite DEEP score or DEEP+ score indicates the risk of prostate cancer progression in the individual; and
d) displaying information regarding the risk of prostate cancer progression in the individual.

27. The computer implemented method of claim 26, further comprising storing the information regarding the risk of prostate cancer progression in the individual in a database.

28. A system for predicting the risk of prostate cancer progression in an individual using the computer implemented method of claim 26 or 27, the system comprising:
a) a storage component for storing data, wherein the storage component has instructions for predicting the risk of prostate cancer progression in an individual based on analysis of the prostate cancer genome sequencing data stored therein;
b) a computer processor for processing the prostate cancer genome sequencing data using one or more algorithms, wherein the computer processor is coupled to the storage component and configured to execute the instructions stored in the storage component in order to receive the inputted prostate cancer genome sequencing data and analyze the data according to the computer implemented method of claim 26 or 27; and
c) a display component for displaying the information regarding the risk of prostate cancer progression in the individual.

29. A non-transitory computer-readable medium comprising program instructions that, when executed by a processor in a computer, causes the processor to perform the computer implemented method of claim 26 or 27.

30. A kit comprising the non-transitory computer-readable medium of claim 29 and instructions for predicting the risk of prostate cancer progression in an individual.

31. A method of diagnosing an individual with prostate cancer, the method comprising:
a) genotyping the individual to determine if the individual has one or more pathogenic non-coding somatic mutations listed in Table 1; and
b) calculating a composite deep estimation from epigenome prediction (DEEP) score or a DEEP+ score for the pathogenic non-coding somatic mutations detected in the individual by genotyping, wherein the composite DEEP score or DEEP+ score indicates whether the individual has prostate cancer.

32. The method of claim 31, further comprising treating the individual for the prostate cancer if the composite DEEP score or DEEP+ score indicates the individual has prostate cancer.

33. The method of claim 32, wherein said treating comprises surgery, radiation therapy, chemotherapy, hormonal therapy, immunotherapy, anti-angiogenic therapy, molecularly targeted or biologic therapy, or photodynamic therapy, or a combination thereof.
